(19) United States
(12) Patent Application Publication
(10) Pub. No.: US 2025/0143848 A1
(43) Pub. Date: May 8, 2025

(54) NON-TRANSITORY COMPUTER-READABLE MEDIUM AND INDEX VALUE CALCULATION APPARATUS

(71) Applicants: Takeshi Matsuoka, Shizuoka (JP); THE NIPPON DENTAL UNIVERSITY, Tokyo (JP)

(72) Inventors: Takeshi Matsuoka, Shizuoka (JP); Masaru Yamakoshi, Shizuoka (JP); Yukihiro Numabe, Tokyo (JP); Hiroshi Ito, Tokyo (JP); Ryutaro Kuraji, Tokyo (JP)

(73) Assignees: Takeshi Matsuoka, Shizuoka (JP); THE NIPPON DENTAL UNIVERSITY, Tokyo (JP)

(21) Appl. No.: 18/925,668

(22) Filed: Oct. 24, 2024

(30) Foreign Application Priority Data
Nov. 2, 2023 (JP) 2023-188628

(51) Int. Cl. A61C 9/00; A61B 5/00; G06T 7/00; G06V 10/25; G16H 30/40; G16H 50/30

(52) U.S. Cl. CPC A61C 9/0053; A61B 5/0088; G06T 7/0012; G06V 10/25; G16H 30/40; G16H 50/30; G06T 2200/04; G06T 2207/30036; G06V 2201/03

(57) ABSTRACT

A program stored in a non-transitory computer-readable medium of the present disclosure causes a computer to execute: acquiring intraoral three-dimensional data that represents a tooth of a subject person and periodontium of the tooth; and calculating a gingival state index value of the subject person, an alveolar bone resorption index value of the subject person, or both thereof as a state index value of the subject person by using the intraoral three-dimensional data. The gingival state index value of the subject person is an index value related to a state of a gingival of the subject person. The alveolar bone resorption index value of the subject person is an index value related to resorption of an alveolar bone of the subject person. The state index value is an index value related to a state of teeth of the subject person.

### INCORPORATION BY REFERENCE

This application is based upon and claims the benefit of priority from Japanese patent application No. 2023-188628, filed on Nov. 2, 2023, the disclosure of which is incorporated herein in its entirety by reference.

### TECHNICAL FIELD

The present disclosure relates to a non-transitory computer-readable medium and an index value calculation apparatus.

### BACKGROUND ART

A technique for predicting and diagnosing a periodontal-related disease with data representing information of an oral cavity has been disclosed. For example, Japanese Unexamined Patent Application Publication No. 2019-030587 discloses a technique of performing a gingivitis inspection by comparing periodontal image data obtained by capturing an oral cavity with reference color data, which is color data of a gingival indicating a state of gingivitis. Japanese Unexamined Patent Application Publication No. 2019-155027 discloses a technique of generating an intraoral image by combining a plurality of partial captured images of a portion of an oral cavity, and determining presence or absence of a prescribed disease and a state in the oral cavity based on features of the image in a predetermined determination target region. Japanese Unexamined Patent Application Publication No. 2021-053175 discloses a technique of presuming a state of an intraoral target region from image data of an intraoral region obtained by capturing an image with a camera, and determining the state of the intraoral target region based on an obtained presumption result and predetermined reference information. Published Japanese Translation of PCT International Publication for Patent Application, No. 2022-508923 discloses a technique of predicting intraoral health by analyzing an intraoral image with a machine learning algorithm and comprehensively analyzing presence or absence of orthodontics, a caries state, a periodontitis state, a prosthetic state, medical questionnaires, and the like. Japanese Unexamined Patent Application Publication No. 2022-073148 discloses a technique of presuming presence or absence of a periodontal disease by inputting a periodontium image of an intraoral image into a model. Japanese Unexamined Patent Application Publication No. 2022-012199 discloses a technique of identifying a part that is likely to cause an intraoral disease by applying three-dimensional data of an intraoral shape to a presumption model including a neural network.

### SUMMARY

Intraoral information includes information of a gingival state and alveolar bone resorption. None of the above-mentioned patent literatures refers to use of three-dimensional data representing an oral cavity for analyzing a gingival state or the alveolar bone resorption. An objective of the present disclosure, which has been made in view of the above problems, is to provide a novel technique for analyzing a state of an oral cavity with three-dimensional data representing the oral cavity.

The present disclosure provides a non-transitory computer-readable medium storing a program causing a computer to execute: acquiring intraoral three-dimensional data that represents a tooth of a subject person and periodontium of the tooth; and calculating a gingival state index value of the subject person, an alveolar bone resorption index value of the subject person, or both thereof as a state index value of the subject person by using the intraoral three-dimensional data. The gingival state index value of the subject person is an index value related to a state of a gingival of the subject person. The alveolar bone resorption index value of the subject person is an index value related to resorption of an alveolar bone of the subject person. The state index value is an index value related to a state of teeth of the subject person.

The present disclosure provides an index value calculation apparatus comprising: at least one memory that is configured to store instructions; and at least one processor that is configured to execute the instructions to: acquire intraoral three-dimensional data that represents a tooth of a subject person and periodontium of the tooth; and calculate a gingival state index value of the subject person, an alveolar bone resorption index value of the subject person, or both thereof as a state index value of the subject person by using the intraoral three-dimensional data. The gingival state index value of the subject person is an index value related to a state of a gingival of the subject person. The alveolar bone resorption index value of the subject person is an index value related to resorption of an alveolar bone of the subject person. The state index value is an index value related to a state of teeth of the subject person.

### BRIEF DESCRIPTION OF DRAWINGS

The above and other aspects, features, and advantages of the present disclosure will become more apparent from the following description of certain example embodiments when taken in conjunction with the accompanying drawings, in which:

FIG. 1 is a diagram illustrating an overview of an operation of an index value calculation apparatus;

FIG. 2 is a view illustrating positions of various parts of a tooth related to a state index value;

FIG. 3 is a block diagram illustrating a functional configuration of the index value calculation apparatus;

FIG. 4 is a block diagram illustrating a hardware configuration of a computer that implements the index value calculation apparatus;

FIG. 5 is a flowchart illustrating a flow of processes executed by the index value calculation apparatus;

FIG. 6 is a flowchart illustrating a flow of processes of calculating a gingival state index value;

FIG. 7 is a diagram illustrating a case in which the gingival state index value is calculated using a gingival state index value calculation model;

FIG. 8 is a diagram illustrating a case in which PPD is calculated using a first prediction model;

FIG. 9 is a diagram illustrating a case in which CAL is calculated using the first prediction model;

FIG. 10 is a diagram illustrating a case in which the gingival state index value is calculated using a first feature value calculation model and the gingival state index value calculation model;

FIG. 11 is a diagram illustrating a case in which PPD is calculated using the first feature value calculation model and the first prediction model;

FIG. 12 is a diagram illustrating a case in which a first feature value calculated without using the first feature value calculation model is used by the gingival state index value calculation model;

FIG. 13 is a diagram illustrating a case in which the first feature value calculated without using the first feature value calculation model is used by the first prediction model;

FIG. 14 is a view representing a case in which a portion of a tooth of interest is included in both a region of interest and a relevant region;

FIG. 15 is a view illustrating a region of interest including only a portion of the tooth of interest and two relevant regions;

FIG. 16 is a diagram illustrating a case in which a gingival state index value 20 is calculated by a gingival state index value calculation model into which a region of interest and a relevant region are input;

FIG. 17 is a diagram illustrating a case in which PPD is calculated using the first prediction model into which the region of interest and the relevant region are input;

FIG. 18 is a flowchart illustrating a flow of processes of calculating an alveolar bone resorption index value;

FIG. 19 is a diagram illustrating a case in which the alveolar bone resorption index value is calculated using an alveolar bone resorption index value calculation model;

FIG. 20 is a diagram illustrating a case in which an alveolar bone resorption bone level is calculated using a second prediction model;

FIG. 21 is a diagram illustrating a case in which the alveolar bone resorption index value is calculated using a third feature value calculation model and the alveolar bone resorption index value calculation model;

FIG. 22 is a diagram illustrating a case in which the alveolar bone resorption bone level is calculated using the third feature value calculation model and the second prediction model;

FIG. 23 is a diagram illustrating a case in which a third feature value calculated without using the third feature value calculation model is used by the alveolar bone resorption index value calculation model;

FIG. 24 is a diagram illustrating a case in which the third feature value calculated without using the third feature value calculation model is used by the second prediction model;

FIG. 25 is a diagram illustrating a case in which the alveolar bone resorption index value is calculated by an alveolar bone resorption index value calculation model into which the region of interest and the relevant region are input;

FIG. 26 is a diagram illustrating a case in which the alveolar bone resorption bone level is calculated using the second prediction model into which the region of interest and the relevant region are input;

FIG. 27 is a block diagram illustrating a functional configuration of the index value calculation apparatus that performs a determination process using an index value;

FIG. 28 is a diagram illustrating training of the gingival state index value calculation model;

FIG. 29 is a diagram illustrating training of the first prediction model;

FIG. 30 is a diagram illustrating training of the first feature value calculation model and the gingival state index value calculation model;

FIG. 31 is a diagram illustrating training of the gingival state index value calculation model into which the first feature value calculated with a predetermined algorithm is input;

FIG. 32 is a diagram illustrating the training of the gingival state index value calculation model using the relevant region;

FIG. 33 is a diagram illustrating the training of the first prediction model using the relevant region;

FIG. 34 is a diagram illustrating training of the alveolar bone resorption index value calculation model;

FIG. 35 is a diagram illustrating training of the second prediction model;

FIG. 36 is a diagram illustrating training of the third feature value calculation model and the alveolar bone resorption index value calculation model;

FIG. 37 is a diagram illustrating the training of the alveolar bone resorption index value calculation model into which the third feature value calculated with a predetermined algorithm is input;

FIG. 38 is a diagram illustrating the training of the alveolar bone resorption index value calculation model using the relevant region; and

FIG. 39 is a diagram illustrating the training of the second prediction model using the relevant region.

### EXAMPLE EMBODIMENT

Hereinafter, embodiments of the present disclosure will be described in detail with reference to the drawings. In each drawing, the same or corresponding elements are denoted with the same signs, and duplicate descriptions are omitted as necessary for clarification of description. Furthermore, unless otherwise described, a value determined in advance such as a predetermined value or a threshold value is stored in advance in a storage apparatus or the like accessible from an apparatus that uses the value. Furthermore, unless otherwise described, a storage unit includes one storage apparatus or an arbitrary number of storage apparatuses.

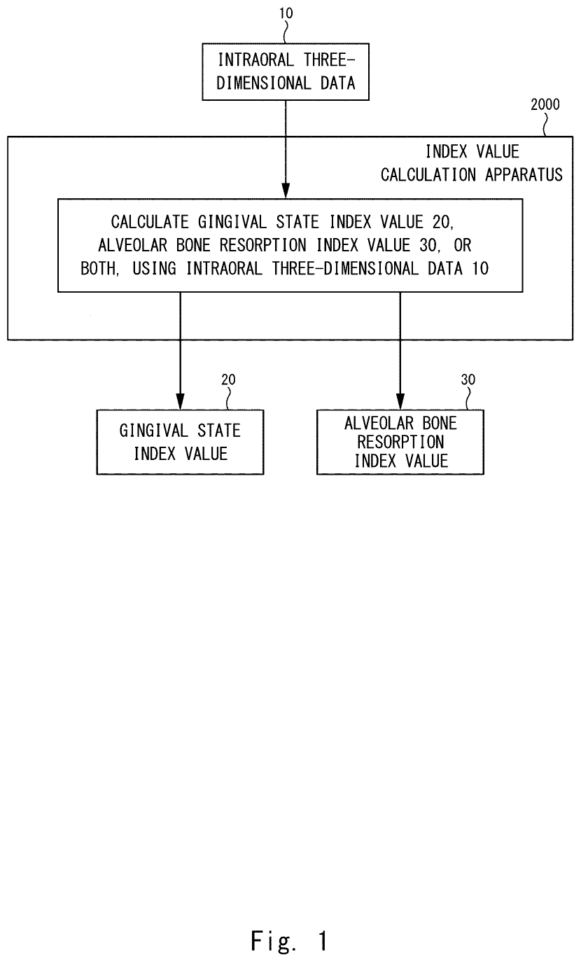

FIG. 1 is a diagram illustrating an overview of an operation of an index value calculation apparatus 2000. Here, FIG. 1 is a diagram for facilitating understanding of the overview of the index value calculation apparatus 2000, and the operation of the index value calculation apparatus 2000 is not limited to the operation shown in FIG. 1.

The index value calculation apparatus 2000 acquires intraoral three-dimensional (3D) data 10. The intraoral 3D data 10 is data representing a 3D shape of a tooth present in an oral cavity of a subject person and a periodontium surface of the tooth. Here, any data capable of representing a 3D shape of an object can be used in the intraoral 3D data 10. Examples of the data representing the 3D shape of the object include a 3D model (for example, voxel data, mesh data, or the like) and point cloud data. The intraoral 3D data 10 may represent distribution of colors in the oral cavity of the subject person in addition to the 3D shape of the oral cavity of the subject person. For example, when the intraoral 3D data 10 is mesh data, the intraoral 3D data 10 represents the 3D shape of the oral cavity of the subject person with a plurality of meshes, and further represents the color distribution in the oral cavity of the subject person by indicating color of each mesh. Note that the intraoral 3D data 10 may include one tooth or a plurality of teeth.

The index value calculation apparatus 2000 calculates a state index value that is an index value of the state of the subject person's teeth, using the intraoral 3D data 10. As the state index value, a gingival state index value 20, an alveolar bone resorption index value 30 or both are calculated. The gingival state index value 20 is an index value representing the state of gingiva. The index value representing a state of the gingiva is, for example, a clinical attachment level (CAL), a probing pocket depth (PPD), presence or absence of bleeding on probing (BOP) or a class of furcation involvement. In addition, the gingival state index value 20 calculated by the index value calculation apparatus 2000 is a predicted value of these index values.

The CAL represents a distance from a cemento-enamel junction (CEJ) to a gingival sulcus floor or pocket bottom that is measured with a periodontal probe, and is an index representing a state of attachment of gingiva to a tooth surface. The PPD represents a distance from a gingival margin to a probe tip during insertion of the periodontal probe, and is an index representing the state of attachment of the gingival to the tooth surface, similarly to the CAL. In general, the probe tip represents the pocket bottom. The CAL and PPD are represented as numerical values in a unit of 1 mm or 0.5 mm, for example.

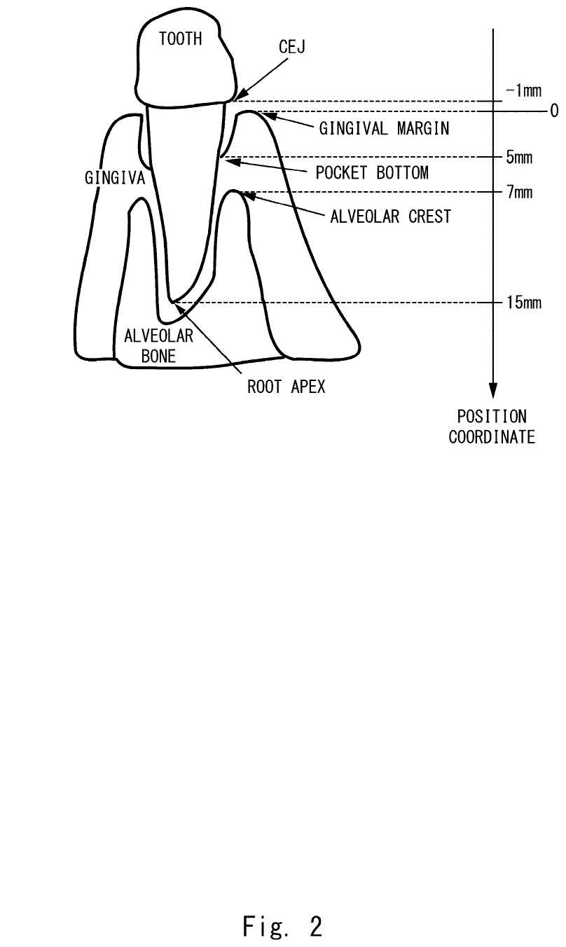

FIG. 2 is a diagram illustrating positions of various parts of the tooth, related to state index values. FIG. 2 shows a gingival margin, CEJ, pocket bottom, alveolar crest, and root apex as parts of the tooth related to the index values. FIG. 2 shows the position of each part as a relative position relative to the position of the gingival margin as a reference (i.e., zero).

For example, the CAL can be represented as a distance from the CEJ to the pocket bottom. In FIG. 2, the positions of the CEJ and the pocket bottom are −1 mm and 5 mm, respectively. Therefore, the CAL is 5−(−1)=6 [mm].

The BOP indicates occurrence of bleeding from the pocket bottom during probing, and based on presence or absence of this bleeding, presence of resistance and inflammation at the bottom of the pocket can be assessed.

The furcation involvement is a state in which lesion caused by a periodontal disease or dental pulp disease has spread to interradicular septum of a multi-rooted tooth (or double rooted tooth). When Lindhe and Hyman's classification of furcation involvement is used, the classification of the furcation involvement is represented by three stage values: first, second, and third stages. When Glickman's classification of furcation involvement is used, the classification of the furcation involvement is represented by four grade values of first, second, third, and fourth grades.

Note that the index value calculation apparatus 2000 may calculate a plurality of types of gingival state index value 20, such as CAL and PPD.

The alveolar bone resorption index value 30 is an index value representing magnitude of alveolar bone resorption. The index value related to the alveolar bone resorption is, for example, a value representing an alveolar bone resorption bone level (BL) or an alveolar bone resorption ratio (ABR ratio). The alveolar bone resorption bone level is calculated from a ratio of a resorbed alveolar bone distance to a root length. Specifically, the alveolar bone resorption bone level is calculated as B/A, wherein A is a distance from the CEJ to a root apex and B is a distance from the CEJ to the alveolar crest. The alveolar bone resorption bone level is represented, for example, by a real number between 0 and 1. The alveolar bone resorption ratio is calculated as B/A*100 [%] using the A and B described above. The alveolar bone resorption index value 30 calculated by the index value calculation apparatus 2000 is a predicted value of these index values.

For example, in FIG. 2, the positions of the CEJ, root apex, and alveolar crest are −1 mm, 15 mm, and 7 mm, respectively. Therefore, in the calculation of the alveolar bone resorption bone level, A=15−(−1)=16 [mm] and B=7−(−1)=8 [mm]. Therefore, the alveolar bone resorption bone level is 8/16=0.5.

In addition, the index value calculation apparatus 2000 may calculate a plurality of types of alveolar bone resorption index values 30, such as the alveolar bone resorption bone level and the alveolar bone resorption ratio.

### <Example of Advantageous Effect>

A periodontal disease, also called a periodontal disorder, is broadly divided into a gingival lesion and periodontitis. The gingival lesion, in particular, plaque-induced gingivitis, is gingival inflammation that develops due to a group of bacteria present at the gingival margin. The periodontitis, in particular, chronic periodontitis, is a chronic inflammatory disease involving attachment loss (loss of adhesion) and alveolar bone resorption caused by periodontal pathogenic bacteria. Accordingly, it is useful to grasp the gingival state and the alveolar bone resorption for diagnosis of a periodontal-related disease.

For example, according to 2018 new classification of the periodontal disease in the American Academy of Periodontology/European Federation of Periodontology, classification of stages I to IV representing severity of periodontitis is based on indices including the CAL, a degree of bone resorption, and the number of teeth lost due to periodontitis. In addition, according to Guideline 2022 for Periodontal Treatment by the Japanese Society of Periodontology, diagnosis of plaque-induced gingivitis and periodontitis is performed along the classification of the periodontal disease based on periodontal tissue inspection or the like. The classification of periodontitis according to a degree of tissue destruction in the diagnosis of one tooth unit is based on an index including the BL, CAL, or presence or absence of the furcation involvement. The classification of periodontitis according to the degree of inflammation is based on an index of PPD. Furthermore, according to a report by the Periodontal Medicine Committee of the Japanese Society of Periodontology, it is suggested as classification of severity recommended by the committee that an alveolar bone resorption ratio of 25% or less is clinically mild, a ratio of 25% to 35% is clinically moderate, and a ratio of 35% or more is clinically sever.

Here, the CAL, PPD, BOP, or furcation involvement, which is an index representing a gingival state, can be determined by measurement with a probe. In addition, the alveolar bone resorption bone level, and the alveolar bone resorption ratio, which are indices representing the resorption of the alveolar bone, can be determined by measurement using an X-ray image. However, there is a problem that the determining by these methods is difficult to conduct by a person other than a person having specialized knowledge and skill (such as a dentist). In addition, there is also a problem that the measurement using the probe is complicated and time-consuming. Furthermore, there is a problem that the measurement using the X-ray image requires expensive and special apparatus and facilities such as an X-ray imaging apparatus and an X-ray imaging room and exposes the subject person to radiation.

In this regard, according to the index value calculation apparatus 2000, the gingival state index value 20 that is the predicted value of the index representing the gingival state, the alveolar bone resorption index value 30 that is the predicted value of the index representing the magnitude of the alveolar bone resorption, or both of them can be calculated using the intraoral 3D data 10. When the gingival state index value 20 is calculated by the index value calculation apparatus 2000, the use of the index value calculation apparatus 2000 allows calculation of the index value representing the gingival state without performing any actual measurement using the probe on the subject person. Accordingly, the use of the index value calculation apparatus 2000 can facilitate the calculation of the gingival state index value 20 even for a person who does not have any specialized knowledge or skill. Furthermore, the use of the index value calculation apparatus 2000 can facilitate the calculation of the gingival state index value 20 without performing any complicated or time-consuming measurement with the probe.

Furthermore, when the index value calculation apparatus 2000 calculates the alveolar bone resorption index value 30, the use of the index value calculation apparatus 2000 allows calculation of the index value representing the resorption of the alveolar bone without performing X-ray imaging of the oral cavity of the subject person. Therefore, the use of the index value calculation apparatus 2000 can facilitate the calculation of the alveolar bone resorption index value 30 even for a person who does not have any specialized knowledge or skill. In addition, with the index value calculation apparatus 2000, the alveolar bone resorption index value 30 can be calculated without using any expensive and special apparatus or facility such as the X-ray imaging apparatus or X-ray imaging room. Furthermore, the index value calculation apparatus 2000 can calculate the alveolar bone resorption index value 30 without concern about exposure to radiation.

Hereinafter, the index value calculation apparatus 2000 of the present embodiment will be described in more detail.

### <Example of Functional Configuration>

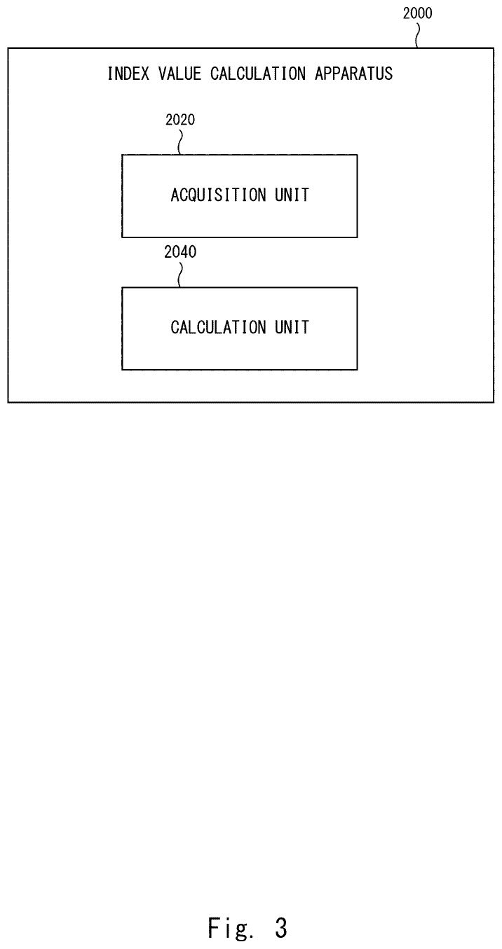

FIG. 3 is a block diagram illustrating a functional configuration of the index value calculation apparatus 2000. The index value calculation apparatus 2000 includes an acquisition unit 2020 and a calculation unit 2040. The acquisition unit 2020 acquires the intraoral 3D data 10. The calculation unit 2040 calculates the gingival state index value 20, the alveolar bone resorption index value 30, or both of them by using the intraoral 3D data 10.

### <Example of Hardware Configuration>

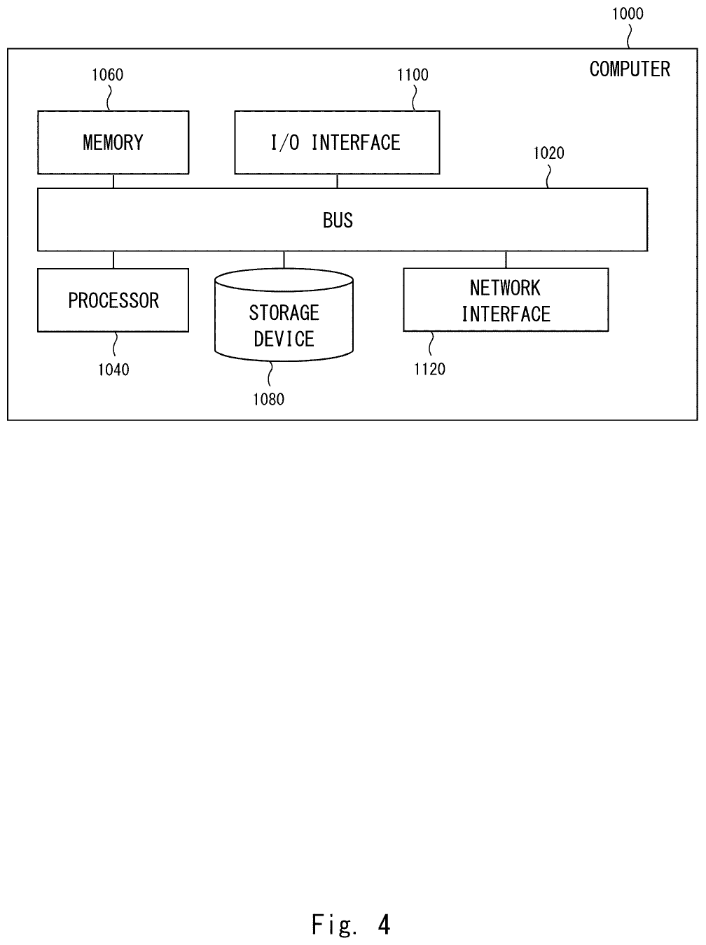

The index value calculation apparatus 2000 may be realized by one or more computers. FIG. 4 is a block diagram illustrating an example of the hardware configuration of a computer 1000 by which the index value calculation apparatus 2000 is implemented.

The computer 1000 may be any type of computer. For example, the computer 1000 is a stationary computer, such as a personal computer (PC) and a server machine. In another example, the computer 1000 is a mobile computer, such as a smartphone and a tablet terminal. In another example, the computer 1000 is an integrated circuit, such as a SoC (system on chip). The computer 1000 may be a special-purpose computer manufactured for implementing the index value calculation apparatus 2000 or may be a general-purpose computer.

The index value calculation apparatus 2000 may be implemented by installing an application in the computer 1000. The application is implemented with a program that causes the computer 1000 to function as the index value calculation apparatus 2000. In other words, the program is an implementation of the functional units of the index value calculation apparatus 2000.

There are various ways to acquire the program. For example, the program may be acquired from a storage medium (e.g., a DVD disk or a USB memory) in which the program is stored. In another example, the program may be downloaded from a server that manages a storage medium storing the program.

In FIG. 4, the computer 1000 includes a bus 1020, a processor 1040, a memory 1060, a storage device 1080, an input/output (I/O) interface 1100, and a network interface 1120. The bus 1020 is a data transmission channel in order for the processor 1040, the memory 1060, the storage device 1080, and the I/O interface 1100, and the network interface 1120 to mutually transmit and receive data. It is noted that the method of connecting these hardware components to each other is not limited to bus connections.

The processor 1040 is a processer, such as a CPU (Central Processing Unit), GPU (Graphics Processing Unit), DSP (Digital Signal Processor), or FPGA (Field-Programmable Gate Array). The memory 1060 is a primary memory component, such as a RAM (Random Access Memory) or a ROM (Read Only Memory). The storage device 1080 is a secondary memory component, such as a hard disk, an SSD (Solid State Drive), or a memory card.

The I/O interface 1100 is an interface between the computer 1000. Peripheral devices, such as a keyboard, mouse, or display device may be connected to the I/O interface 1100.

The network interface 1120 is an interface between the computer 1000 and a network. The network may be a LAN (Local Area Network) or a WAN (Wide Area Network).

The storage device 1080 stores a program that implements each functional configuration of the index value calculation apparatus 2000, i.e., the program that implements the application mentioned above. The processor 1040 may be configured to load instructions of the above-mentioned program from the storage device 1080 into the memory 1060 and execute those instructions, so as to cause the computer 1000 to operate as the index value calculation apparatus 2000.

The hardware configuration of the computer 1000 is not restricted to that shown by FIG. 4. For example, as mentioned-above, the index value calculation apparatus 2000 may be realized as a combination of multiple computers. In this case, those computers may be connected with each other through the network.

The index value calculation apparatus 2000 may be installed in a place where an image of the oral cavity of the subject person is captured, such as a medical examination facility, or may be installed in a place other than the place where the image of the oral cavity of the subject person is captured. In the latter case, for example, the index value calculation apparatus 2000 can be implemented as a server apparatus or the like configured to receive the intraoral 3D data 10 from a terminal that has been used to capture the oral cavity of the subject person.

### <Flow of Process>

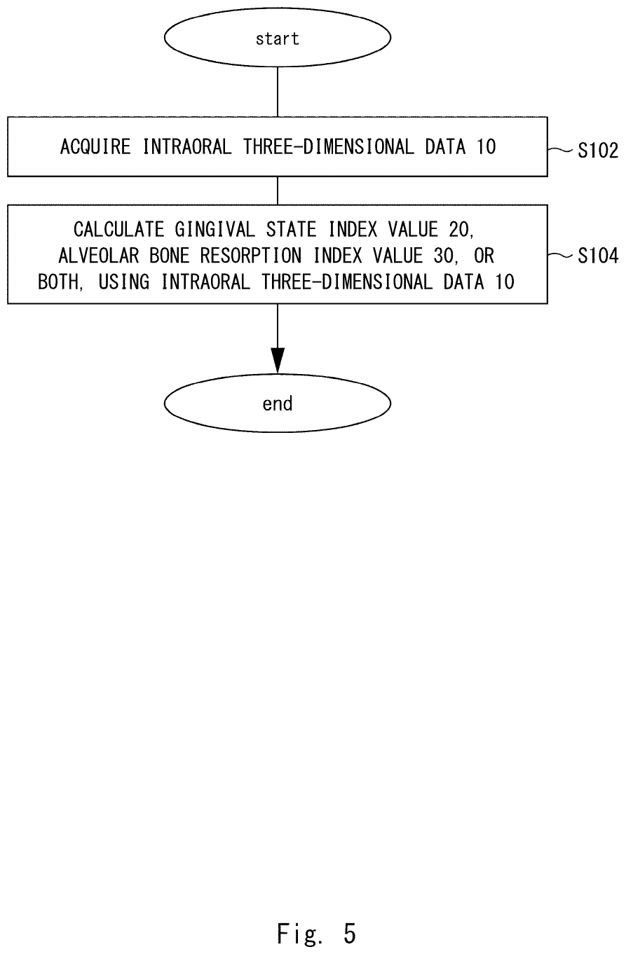

FIG. 5 is a flowchart illustrating a flow of processes executed by the index value calculation apparatus 2000. The acquisition unit 2020 acquires the intraoral 3D data 10 (S102). The calculation unit 2040 calculates the gingival state index value 20, the alveolar bone resorption index value 30, or both of them by using the intraoral 3D data 10 (S104).

Here, in the case where the calculation unit 2040 calculates both the gingival state index value 20 and the alveolar bone resorption index value 30, the calculation of the gingival state index value 20 and the calculation of the alveolar bone resorption index value 30 can be executed in an arbitrary order. Furthermore, the calculation of the gingival state index value 20 and the calculation of the alveolar bone resorption index value 30 may be executed in parallel.

<Method of Generating Intraoral 3D data 10>

The intraoral 3D data 10 is generated by capturing a tooth of the subject person and periodontium of the tooth with an arbitrary image capturing apparatus. In addition, an existing technique can be used for a technique of generating data representing the 3D shape and color distribution of an object with the image capturing apparatus.

As the image capturing apparatus for use in generating the intraoral 3D data 10, various image capturing apparatuses can be adopted, such as a camera of a smartphone, a general-purpose digital camera, a dental digital camera, a camera for dental intraoral imaging, a 3D scanner, an intraoral 3D scanner, and a dental 3D scanner. Furthermore, the image capturing apparatus is not limited to an apparatus capturing visible light, and may be an apparatus capturing near-infrared light, or an apparatus capturing reflected light including a single or plurality of wavelengths during irradiation with a single wavelength of visible to near-infrared light.

As a light source for use in capturing the teeth of the subject person, any light source such as sunlight, incandescent lamp, fluorescent lamp, LED, laser light, or near-infrared light source can be used. Here, an amount of light radiated in the oral cavity may be adjusted using a grating or wavelength selection filter.

Furthermore, the intraoral 3D data 10 may be generated using a ranging apparatus such as light detection and ranging (LiDAR). For example, by scanning the oral cavity of the subject person with the ranging apparatus, point cloud data representing the 3D shape of the oral cavity of the subject person can be obtained. In addition, a 3D model such as mesh data can be generated using the point cloud data obtained in this manner.

### <Acquisition of Intraoral 3D Data 10: S102>

The acquisition unit 2020 acquires the intraoral 3D data 10 (S102). There are various methods of acquiring, by the acquisition unit 2020, the intraoral 3D data 10. For example, the intraoral 3D data 10 is stored in advance in a storage unit accessible from the index value calculation apparatus 2000. In this case, the acquisition unit 2020 acquires the intraoral 3D data 10 from this storage unit.

When a plurality of intraoral 3D data 10 is stored in the storage unit, for example, the acquisition unit 2020 accepts designation of the intraoral 3D data 10 to be used from a user of the index value calculation apparatus 2000. The acquisition unit 2020 acquires the intraoral 3D data 10 designated by the user among the plurality of intraoral 3D data 10 stored in the storage unit.

Here, the storage unit storing the intraoral 3D data 10 may be a storage unit (for example, the storage device 1080) provided inside the index value calculation apparatus 2000, or a storage unit provided outside the index value calculation apparatus 2000. For example, in the case where an image capturing apparatus that generates the intraoral 3D data 10 is provided in the index value calculation apparatus 2000, the intraoral 3D data 10 generated by the image capturing apparatus may be stored in the storage unit inside the index value calculation apparatus 2000. Then, the index value calculation apparatus 2000 acquires the intraoral 3D data 10 stored in the storage unit inside the index value calculation apparatus 2000.

The acquisition unit 2020 may acquire the intraoral 3D data 10 by receiving the intraoral 3D data 10 transmitted from another apparatus. In this case, for example, the intraoral 3D data 10 is transmitted from a terminal (hereinafter referred to as a user terminal) operated by the user of the index value calculation apparatus 2000 to the index value calculation apparatus 2000.

In another example, the acquisition unit 2020 may acquire one or more images of the oral cavity of the subject person from the image capturing apparatus, and generate the intraoral 3D data 10 using the acquired image. Here, the image capturing apparatus may be provided in the user terminal. In this case, for example, the user operates the user terminal and causes the image capturing apparatus to capture the oral cavity of the subject person, thereby causing the image capturing apparatus to generate a captured image of the oral cavity of the subject person. Thereafter, the user further operates the user terminal and transmits the above-described image from the user terminal to the index value calculation apparatus 2000.

The acquisition unit 2020 may apply predetermined image processing to the image obtained from the image capturing apparatus and generate the intraoral 3D data 10 using the image to which the image processing is applied. For example, the acquisition unit 2020 acquires an image generated by capturing the image of the oral cavity of the subject person via no filter and applies predetermined filter processing to this image. Then, the acquisition unit 2020 generates the intraoral 3D data 10 using the image to which the filter processing is applied. As the filter processing, for example, wavelength selection filter application processing for obtaining an image only in a determined wavelength region, polarization filter application processing for reducing effects of reflection, and the like can be adopted.

### <Calculation of State Index Value: S104>

The calculation unit 2040 calculates the gingival state index value 20, the alveolar bone resorption index value 30, or both of them using the intraoral 3D data 10 (S104). Hereinafter, a method of calculating the gingival state index value 20 and a method of calculating the alveolar bone resorption index value 30 will be described, respectively.

### <<Method of Calculating Gingival State Index Value 20>>

For example, the calculation unit 2040 extracts, from the intraoral 3D data 10, a region of interest (ROI) that is a 3D region including a tooth and periodontium of the tooth, and uses the ROI to calculate the gingival state index value 20. Hereinafter, the tooth and the periodontium of the tooth that are prediction targets of the state index value are referred to as the tooth of interest and a periodontium of interest, respectively.

The ROI includes a part that is a prediction target of the state index value. When the entire tooth of interest is the prediction target of the state index value, the ROI includes the tooth of interest and the periodontium of interest. When a portion of the tooth of interest is the prediction target of the state index value, the ROI includes the portion of the tooth of interest and the periodontium of the portion. In addition, in the case where the entire intraoral 3D data 10 represents one tooth and the periodontium of the tooth (in other words, there is one tooth included in the intraoral 3D data 10) and in the case where the entire one tooth is the prediction target of the state index value, the calculation unit 2040 may handle the entire intraoral 3D data 10 as one ROI.

In the case where the intraoral 3D data 10 includes a plurality of teeth, for example, the calculation unit 2040 extracts an ROI for each of all teeth included in the intraoral 3D data 10. In another example, the calculation unit 2040 may extract an ROI only from each of one or more specific teeth among the plurality of teeth included in the intraoral 3D data 10. For example, the calculation unit 2040 extracts the ROI only for a tooth closest to a specific position (for example, a center position) of the intraoral 3D data 10 among the plurality of teeth included in the intraoral 3D data 10. In another example, the calculation unit 2040 extracts an ROI only for the tooth determined by the user among the plurality of teeth included in the intraoral 3D data 10.

As described above, the prediction target of the state index value may be a portion of the tooth. In this case, the calculation unit 2040 may extract a plurality of ROIs for one tooth. For example, the calculation unit 2040 detects a 3D region representing the tooth and the periodontium of the tooth from the intraoral 3D data 10 and divides the detected 3D region according to a predetermined division rule, to extract a plurality of ROIs for one tooth.

Here, various rules can be adopted for the division rule. For example, the division rule is a rule of “dividing the region into a buccal region and a lingual region”. In another example, the division rule is a rule of “dividing the region into a mesial part and a distal part”. Here, the mesial part means a portion of the tooth and the periodontium of the tooth on a side closer to a midline (a side that is away from back teeth). The distal part means a portion of the tooth and the periodontium of the tooth away from the midline (a side that is closer to the back teeth).

The division rule may be a rule of dividing a 3D region of the tooth and gingiva around the tooth into three or more (e.g., four or six) ROIs. For example, the division rule may be a rule of “dividing the region into a predetermined number of regions in a horizontal direction” or “dividing the region into a predetermined number of regions in a vertical direction”. Furthermore, as another division rule, a rule of extracting one or more 3D regions having a predetermined shape (rectangular cuboid, cube, sphere, or ellipsoid, or the like) may be used.

The calculation unit 2040 may use a plurality of division rules. For example, the calculation unit 2040 uses two division rules including the division rule of “dividing the region into the buccal region and the lingual region” and the division rule of “dividing the region into the mesial part and the distal part”. In this case, the calculation unit 2040 performs both of the process of dividing the 3D region representing the tooth and the periodontium of the tooth into a buccal side and a lingual side, and the process of dividing the region into the mesial part and the distal part. Thus, four ROIs can be obtained for one tooth.

The extraction of the ROI is performed, for example, using a machine learning model trained to extract the ROI from the intraoral 3D data 10. Hereinafter, a model that extracts the ROI from the intraoral 3D data 10 is referred to as an ROI extraction model. For the ROI extraction model, any machine learning model capable of extracting a predetermined 3D region from 3D data (for example, a neural network such as a convolutional neural network (CNN)) can be used.

The ROI extraction model is configured to output one or more 3D regions to be treated as the ROI, for example, in response to the input of the intraoral 3D data 10.

The model may not be used for extracting the ROI. For example, the calculation unit 2040 may analyze the intraoral 3D data 10 with a predetermined algorithm, to extract the ROI from the intraoral 3D data 10.

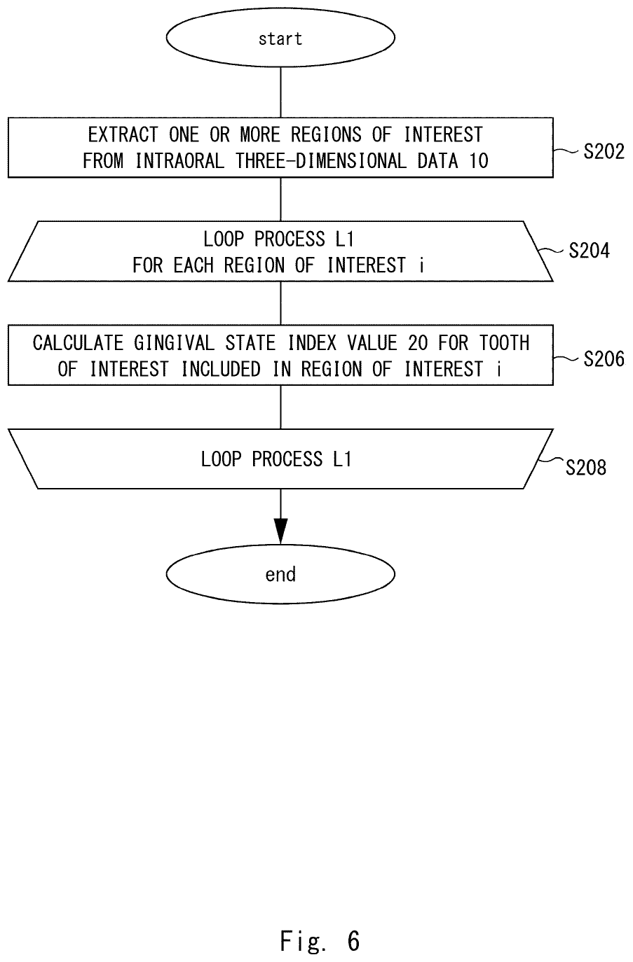

FIG. 6 is a flowchart illustrating a flow of processes of calculating the gingival state index value 20. The calculation unit 2040 extracts one or more ROIs from the intraoral 3D data 10 (S202). Steps S204 to S208 constitute a loop process L1 executed for each ROI. In S204, the calculation unit 2040 determines whether the loop process L1 has been executed for all ROIs. In a case where the loop process L1 has already been executed for all the ROIs, the processing of FIG. 6 ends.

If there is one or more ROIs that are not yet subjected to the loop process L1, the calculation unit 2040 selects one of the ROIs that are not yet subjected to the loop process L1. The ROI selected here is denoted as an ROI i.

The calculation unit 2040 calculates the gingival state index value 20 for the ROI i (S206). S208 is the end of the loop process L1, and hence S204 is executed again.

The process (S206) of calculating the gingival state index value 20 from the ROI is performed, for example, using a trained machine learning model. Hereinafter, a model that calculates the gingival state index value 20 is referred to as a gingival state index value calculation model.

For the gingival state index value calculation model, for example, any model such as a logistic regression model, a multiple regression model, multilayer perceptron, a neural network such as CNN or a recurrent neural network (RNN), a support vector machine, a random forest modeled as a regression tree, or a hidden Markov model can be adopted. In addition, a model that combines various models and makes comprehensive determination can be adopted as the gingival state index value calculation model.

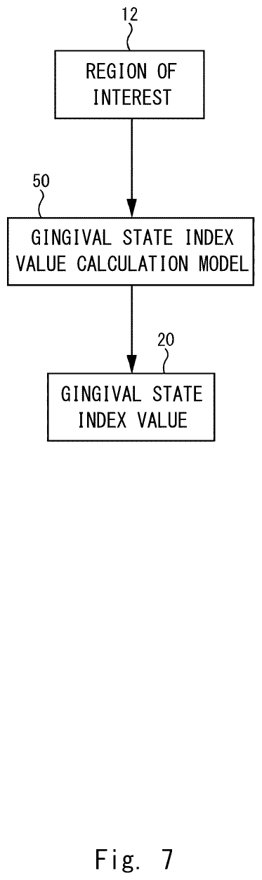

FIG. 7 is a diagram illustrating a case in which the gingival state index value 20 is calculated using a gingival state index value calculation model. In FIG. 7, a gingival state index value calculation model 50 is configured to output the gingival state index value 20 in response to input of an ROI 12.

The calculation unit 2040 inputs the ROI 12 extracted from the intraoral 3D data 10 into the gingival state index value calculation model 50. Thus, the calculation unit 2040 obtains the gingival state index value 20 for the ROI 12 from the gingival state index value calculation model 50.

Here, when the calculation unit 2040 calculates a plurality of types of gingival state index values 20, the calculation unit 2040 includes the gingival state index value calculation model 50 for each type of gingival state index value 20. Suppose that CAL and PPD are calculated as gingival state index values 20. In this case, the calculation unit 2040 includes the gingival state index value calculation model 50 trained to calculate the CAL and the gingival state index value calculation model 50 trained to calculate the PPD.

Furthermore, the calculation unit 2040 may include the gingival state index value calculation model 50 for each tooth or tooth part. Here, suppose that the number of teeth is N. In the case where the gingival state index value 20 is calculated for each tooth, the calculation unit 2040 includes N gingival state index value calculation models 50 for each type of index value calculated as the gingival state index value 20. For example, in the case where the PPD and CAL are calculated as the gingival state index values 20, the calculation unit 2040 includes 2*N gingival state index value calculation models 50.

Furthermore, in the case where an index value is calculated for M parts of each tooth, the calculation unit 2040 includes M*N gingival state index value calculation models 50 for each type of index value calculated as the gingival state index value 20. For example, in the case where the PPD and CAL are calculated as the gingival state index values 20, the calculation unit 2040 includes 2*M*N gingival state index value calculation models 50.

In addition, in consideration of left-right symmetry via an oral midline as a reference, the number of gingival state index value calculation models 50 may be set to half of the number described above. In this case, the same gingival state index value calculation model 50 is used for two ROIs 12 that are located at left and right symmetrical positions via the oral midline, respectively.

Preparing the gingival state index value calculation model 50 for each tooth or tooth part has an advantage that the gingival state index value 20 can be calculated with higher accuracy than in a case of using the gingival state index value calculation model 50 common to all teeth and tooth parts.

The gingival state index value calculation model 50 may not be used in calculating the gingival state index value 20. For example, the calculation unit 2040 may be configured to predict a value of a parameter required for calculating the gingival state index value 20 using a machine learning model. Hereinafter, a model that predicts the value of the parameter required for calculating the gingival state index value 20 is referred to as a first prediction model. A type of machine learning model that can be used as the first prediction model is the same as a type of machine learning model that can be used as the gingival state index value calculation model 50.

The first prediction model is, for example, trained in advance to output a value of a specific parameter in response to input of the ROI 12. The calculation unit 2040 inputs the ROI 12 into the first prediction model and uses the value of the parameter obtained from the first prediction model, to calculate the gingival state index value 20.

The parameter required for calculating the gingival state index value 20 varies with a type of index value calculated as the gingival state index value 20. Suppose that PPD is calculated as the gingival state index value 20. The PPD is a distance between the gingival margin and the pocket bottom. Accordingly, it is necessary for calculating the PPD to determine a position of the gingival margin and a position of the pocket bottom.

Therefore, the calculation unit 2040 includes, for example, a first prediction model that is trained to predict the position of the gingival margin for the ROI 12 and a first prediction model that is trained to predict the position of the pocket bottom for the ROI 12. The calculation unit 2040 calculates the PPD using the first prediction models.

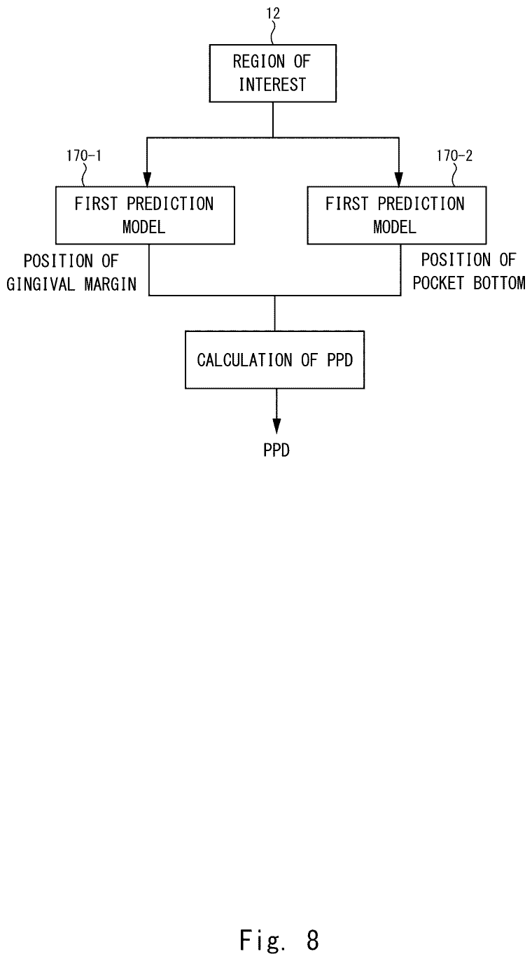

FIG. 8 is a diagram illustrating a case in which PPD is calculated using the first prediction model. A first prediction model 170-1 is the first prediction model that predicts the position of the gingival margin, while a first prediction model 170-2 is the first prediction model that predicts the position of the pocket bottom.

The calculation unit 2040 inputs the ROI 12 into the first prediction model 170-1, to obtain the position of the gingival margin. Furthermore, the calculation unit 2040 inputs the ROI 12 into the first prediction model 170-2, to obtain the position of the pocket bottom. The calculation unit 2040 then calculates a difference between the position of the gingival margin obtained from the first prediction model 170-1 and the position of the pocket bottom obtained from the first prediction model 170-2, to calculate the PPD.

Suppose that a CAL is calculated as the gingival state index value 20. The CAL is a distance between the CEJ and the pocket bottom. It is necessary for calculating the CAL to determine a position of the CEJ and the position of the pocket bottom.

For example, the calculation unit 2040 includes a first prediction model 170 that is trained to predict the position of the CEJ for the ROI 12 and a first prediction model 170 that is trained to predict the position of the pocket bottom for the ROI 12. The calculation unit 2040 uses the first prediction models 170 to calculate the CAL.

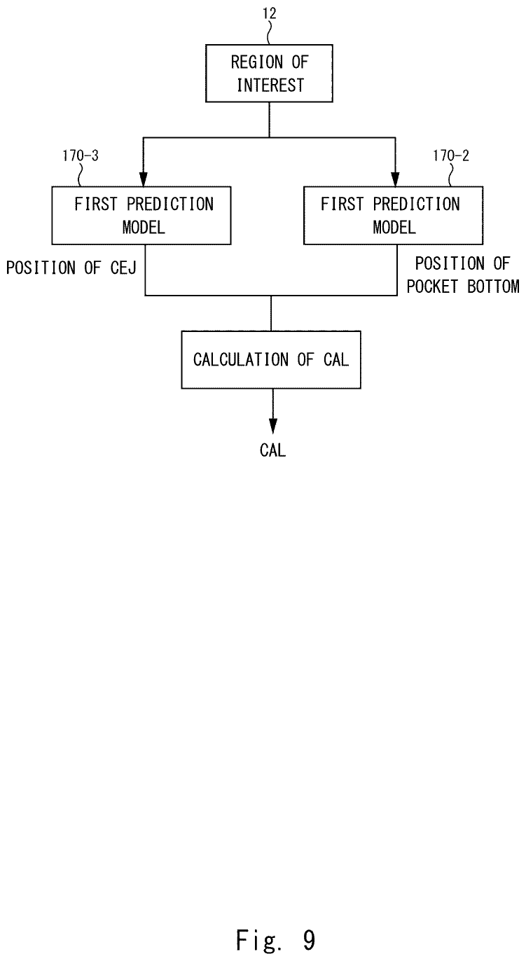

FIG. 9 is a diagram illustrating a case in which a CAL is calculated using the first prediction model. A first prediction model 170-3 is a first prediction model that predicts the position of the CEJ, while the first prediction model 170-2 is the first prediction model that predicts the position of the pocket bottom.

The calculation unit 2040 inputs the ROI 12 into the first prediction model 170-3, to obtain the position of the CEJ. Furthermore, the calculation unit 2040 inputs the ROI 12 into the first prediction model 170-2, to obtain the position of the pocket bottom. The calculation unit 2040 then calculates a difference between the position of the CEJ obtained from the first prediction model 170-3 and the position of the pocket bottom obtained from the first prediction model 170-2, to calculate the CAL.

It is noted that the pocket bottom is hidden by the gingiva (see FIG. 2). By using the first prediction model 170, the calculation unit 2040 can predict the position of the pocket bottom hidden by the gingiva. In addition, there is also a case in which the CEJ is hidden by the gingiva. By using the first prediction model 170, the calculation unit 2040 can predict the position of the CEJ even if the CEJ is hidden by the gingiva. Therefore, the use of the first prediction model 170 can facilitate calculation of the index value requiring the position of a part hidden by the gingiva.

The calculation unit 2040 includes the first prediction model 170 for each parameter to use it to calculate the gingival state index value 20, for example. For example, when the PPD and CAL are calculated as the gingival state index values 20, the calculation unit 2040 includes the first prediction model 170 that predicts the position of the gingival margin, the first prediction model 170 that predicts the position of the pocket bottom, and the first prediction model 170 that predicts the position of the CEJ.

The first prediction model 170 may be prepared for each tooth or tooth part. For example, in the case where the number of teeth is N, the calculation unit 2040 includes N first prediction models 170 for each parameter. For example, in the case where the PPD and CAL are calculated as the gingival state index values 20, the calculation unit 2040 includes N first prediction models 170 that predict the position of the gingival margin, N first prediction models 170 that predict the position of the pocket bottom, and N first prediction models 170 that predict the position of the CEJ.

Furthermore, in the case where an index value is calculated for M parts of each tooth, the calculation unit 2040 includes M*N first prediction models 170 for each parameter. For example, in the case where the PPD and CAL are calculated as the gingival state index values 20, the calculation unit 2040 includes N*M first prediction models 170 that predict the position of the gingival margin, N*M first prediction models 170 that predict the position of the pocket bottom, and N*M first prediction models 170 that predict the position of the CEJ.

In consideration of left-right symmetry via the oral midline as the reference, the number of gingival state index value calculation models 50 may be set to half of the number described above. In this case, the same first prediction models 170 is used for two ROIs 12 that are located at left and right symmetrical positions via the oral midline.

Preparing the first prediction model 170 for each tooth or tooth part as mentioned above has an advantage that the value of the parameter can be predicted with higher accuracy than in a case of using the first prediction model 170 common to all teeth and tooth parts.

The data that is input to the gingival state index value calculation model 50 and to the first prediction model 170 may be a feature value that can be calculated from the ROI 12, rather than the ROI 12. Hereinafter, the feature value that is calculated from the ROI 12 and used to calculate the gingival state index value 20 is referred to as a first feature value.

The first feature value is, for example, a value representing a predetermined type of feature related to a tooth of interest, or a value representing a predetermined type of feature related to periodontium of interest. Features related to the tooth include, for example, a shape of the tooth, a size of the tooth, a tone of the tooth (change in tooth color density depending on the position), smoothness of the tooth, a distance between the tooth and the adjacent tooth (a size of a gap), and presence or absence of CEJ exposure in the tooth. It is noted that a feature of a size of an object is represented, for example, by a vertical length of the object, a lateral length of the object, a thickness of the object, or the like. Furthermore, a feature of a shape of the object is represented, for example, by a ratio of the vertical length of the object to the lateral length of the object, a ratio of a width of an upper portion of the object to a width of a lower portion of the object, or the like.

The smoothness includes concepts such as a degree of smoothness, a degree of roughness, and a degree of unevenness. The smoothness of the tooth can be represented, for example, by a smoothness rate. The smoothness rate is represented, for example, by an area ratio of a region in which deviation from an approximated surface obtained by approximating the surface of the tooth with an elliptical surface is within a reference value.

Features related to the periodontium include, for example, a color of gingiva, a tone of the gingiva (change in color density of the gingiva depending on the position), a shape of the gingiva, a surface smoothness of the gingiva, a distance between the gingiva surface and the tooth surface (degree of protrusion of the gingiva from the tooth), a surface area of the gingiva, a volume of the gingiva, a distance between gingival alveolar mucosal border and gingival margin, a shape of gingival papilla, a surface area of the gingival papilla, a volume of the gingival papilla volume, a height of the gingival papilla, or the like.

Here, a value represented by the first feature value may be an absolute value or may be a relative value to a reference value. The reference value may be obtained from the intraoral 3D data 10 or may be defined in advance. When the reference value is obtained from the intraoral 3D data 10, for example, an object representing a reference is captured together with teeth of the subject person by the image capturing apparatus.

The first feature value is calculated, for example, using a machine learning model trained in advance. Hereinafter, the model for use in calculating the first feature value is referred to as a first feature value calculation model. A type of model that can be used as the first feature value calculation model is the same as a type of model that can be used as the gingival state index value calculation model.

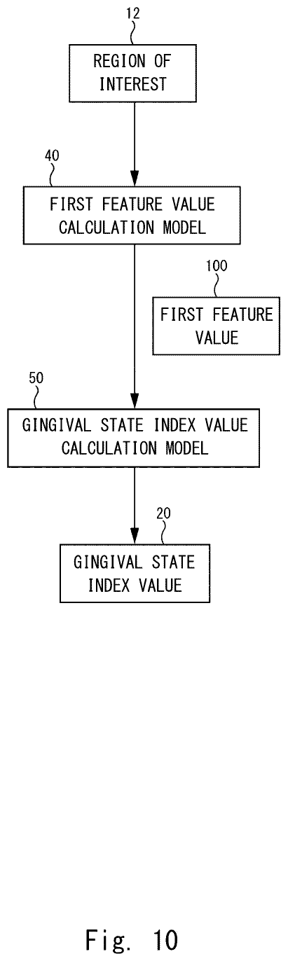

FIG. 10 is a diagram illustrating a case in which the gingival state index value 20 is calculated using the first feature value calculation model and the gingival state index value calculation model. A first feature value calculation model 40 is trained in advance to output a first feature value 100 in response to the input of the ROI 12. Furthermore, the gingival state index value calculation model 50 is trained in advance to output the gingival state index value 20 in response to the input of the first feature value 100.

The calculation unit 2040 inputs the ROI 12 extracted from the intraoral 3D data 10 into the first feature value calculation model 40. Furthermore, the calculation unit 2040 inputs the first feature value 100 output from the first feature value calculation model 40 into the gingival state index value calculation model 50. Thus, the calculation unit 2040 obtains the gingival state index value 20 for the ROI 12 from the gingival state index value calculation model 50.

Here, in the case where the calculation unit 2040 includes a plurality of gingival state index value calculation models 50, the first feature value calculation model 40 may be shared by all the gingival state index value calculation models 50, or the first feature value calculation model 40 may be prepared for each gingival state index value calculation model 50.

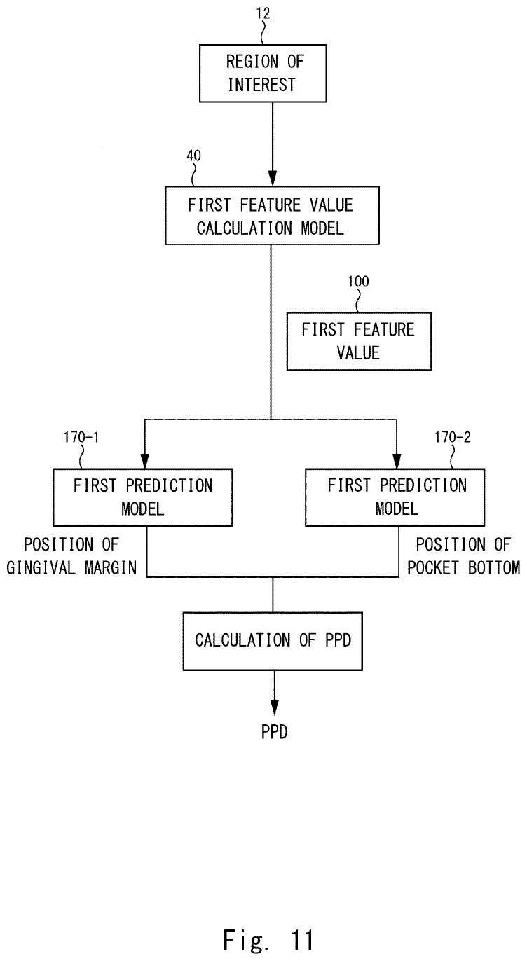

FIG. 11 is a diagram illustrating a case in which PPD is calculated using a first feature value calculation model and a first prediction model. The first prediction model 170-1 is trained in advance to output the position of the gingival margin in response to the input of the first feature value 100. The first prediction model 170-2 is trained in advance to output the position of the pocket bottom in response to the input of the first feature value 100.

The calculation unit 2040 inputs the ROI 12 extracted from the intraoral 3D data 10 into the first feature value calculation model 40. Furthermore, the calculation unit 2040 inputs the first feature value 100 output from the first feature value calculation model 40 into each of the first prediction models 170-1 and 170-2. The calculation unit 2040 then calculates a difference between the position of the gingival margin obtained from the first prediction model 170-1 and the position of the pocket bottom obtained from the first prediction model 170-2, to calculate the PPD.

Here, in the case where the calculation unit 2040 includes a plurality of first prediction models 170, the first feature value calculation model 40 may be shared by all the first prediction models 170, or the first feature value calculation model 40 may be prepared for each first prediction model 170.

The first feature value calculation model 40 may not be used in calculating the first feature value 100. In this case, the calculation unit 2040 analyzes the ROI 12 with a predetermined algorithm, to calculate the first feature value 100 from the ROI 12.

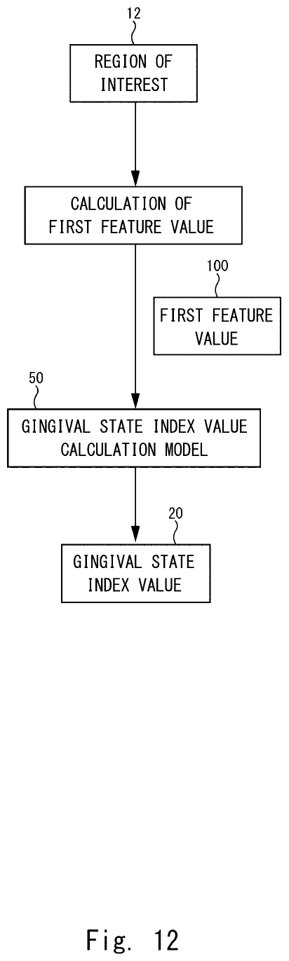

FIG. 12 is a diagram illustrating a case in which the first feature value calculated without using the first feature value calculation model 40 is used by the gingival state index value calculation model 50. The calculation unit 2040 analyzes the ROI 12, to calculate the first feature value 100. Then, the calculation unit 2040 inputs the calculated first feature value 100 into the gingival state index value calculation model 50. By doing so, the gingival state index value 20 is obtained from the gingival state index value calculation model 50.

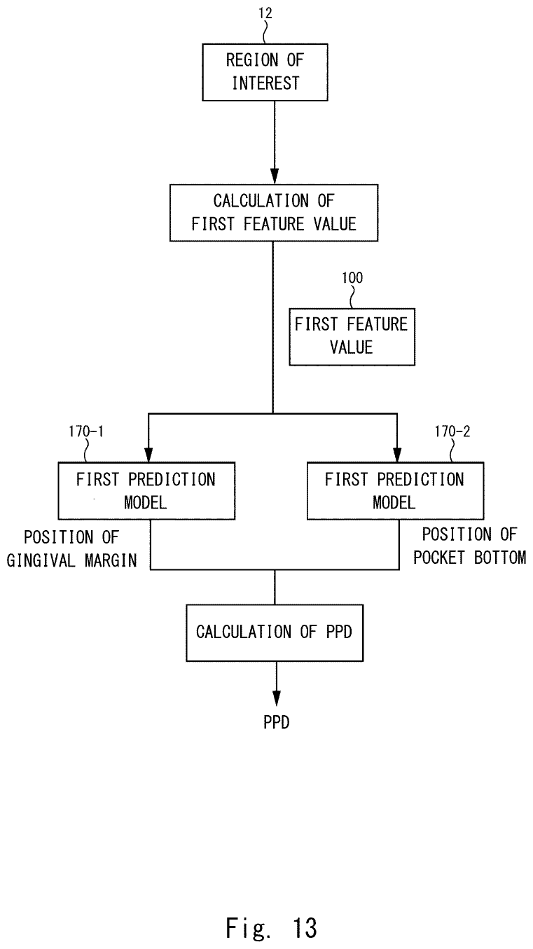

FIG. 13 is a diagram illustrating a case in which the first feature value calculated without using the first feature value calculation model 40 is used by the first prediction model 170. In an example of FIG. 13, the PPD is calculated as the gingival state index value 20.

The calculation unit 2040 analyzes the ROI 12, to calculate the first feature value 100. Thereafter, the calculation unit 2040 inputs the calculated first feature value 100 to each of the first prediction models 170-1 and 170-2. The calculation unit 2040 then calculates a difference between the position of the gingival margin obtained from the first prediction model 170-1 and the position of the pocket bottom obtained from the first prediction model 170-2, to calculate the PPD.

In calculating the gingival state index value 20 from the ROI 12, a 3D region other than the ROI 12 may be further used. Specifically, the calculation unit 2040 may further use another 3D region (hereinafter referred to as the relevant region) associated with the ROI 12 among 3D regions included in the intraoral 3D data 10. The relevant region is, for example, a region proximal to the ROI. In another example, the relevant region is a region including a tooth at the position symmetrical to the tooth of interest via the oral midline as a reference.

The region proximal to the ROI is, for example, a region including a tooth (hereinafter referred to as a proximal tooth) that is proximal to the tooth of interest. The proximal tooth is, for example, a tooth adjacent to the tooth of interest. A tooth adjacent to the tooth of interest is a tooth on a mesial side or a distal side of the tooth of interest. The proximal tooth may be a tooth located at a position of a predetermined number of (e.g., two) teeth away from the tooth of interest.

Suppose that the ROI includes only a portion of the tooth of interest, rather than the entire tooth of interest. In this case, the relevant region may be a region including a portion of the tooth of interest that is not included in the ROI. For example, the relevant region is a region located at a position symmetrical to a target region via a tooth centerline of the tooth of interest as a reference.

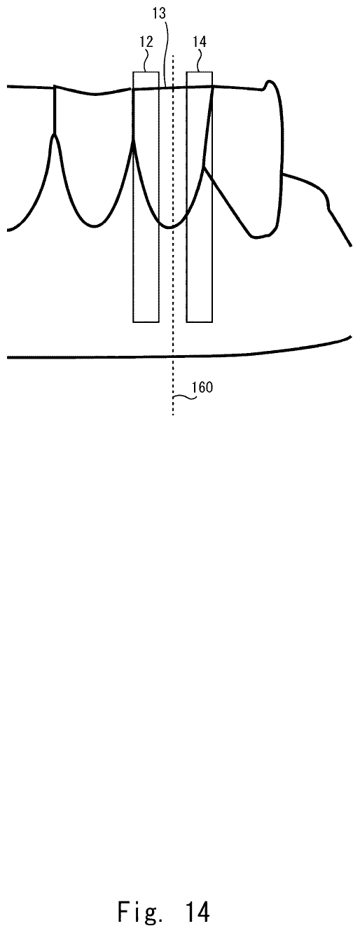

FIG. 14 is a view illustrating a case in which a portion of the tooth of interest is included in both the ROI and the relevant region. In FIG. 14, the ROI 12 includes a tooth of interest 13 and periodontium of the tooth. The relevant region 14 is a region that is located at a position symmetrical to the ROI 12 via a tooth centerline 160 and includes the tooth of interest 13 and periodontium of the tooth.

The relevant region 14 may have the same size as a size of the ROI 12, may have a size larger than the size of the ROI 12, or may have a size smaller than the size of the ROI 12. If the size of the relevant region 14 is different from the size of the ROI 12, for example, the size of the relevant region 14 is set to a predetermined multiple (e.g., two times or the like) of the size of the ROI 12.

Here, in the relevant region 14, the size set to the predetermined multiple of the size of the ROI 12 may be a length along each axial direction or a length only along a certain axial direction. In the latter case, for example, the size of the relevant region 14 is set to the predetermined multiple of the size of the ROI 12, only for a lateral length (length along a horizontal direction in FIG. 14) in the case where the teeth are viewed from front. For remaining axial lengths, the size of the relevant region 14 is set to the same size as the size of the ROI 12.

The calculation unit 2040 may use two or more relevant regions 14. For example, the calculation unit 2040 uses, as the relevant region 14, a region of respective proximal teeth having a distance within two teeth from the tooth of interest.

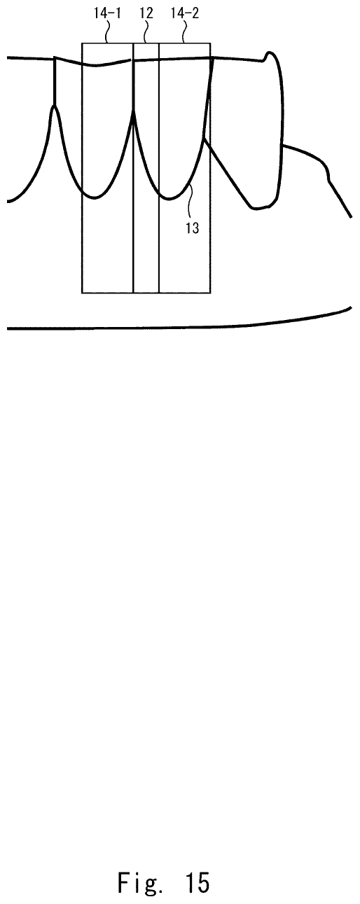

Suppose that the ROI includes only a portion of the tooth of interest, rather than the entire tooth of interest. In this case, for example, as the relevant region 14, a relevant region 14 adjacent from the mesial side to the ROI and a relevant region 14 adjacent from the distal side to the ROI can be used. FIG. 15 is a view illustrating an ROI 12 including only a portion of the tooth of interest and two relevant regions 14. In an example of FIG. 15, two relevant regions 14 of relevant regions 14-1 and 14-2 are used for one ROI 12. In FIG. 15, a size of the relevant region 14 is set to twice a size of the ROI 12.

The relevant region 14 is used by the gingival state index value calculation model 50 and the first prediction model 170. Suppose that the gingival state index value 20 is calculated using the gingival state index value calculation model 50. In this case, the gingival state index value calculation model 50 is trained in advance to output the gingival state index value 20 in response to the input of both the ROI 12 and the relevant region 14.

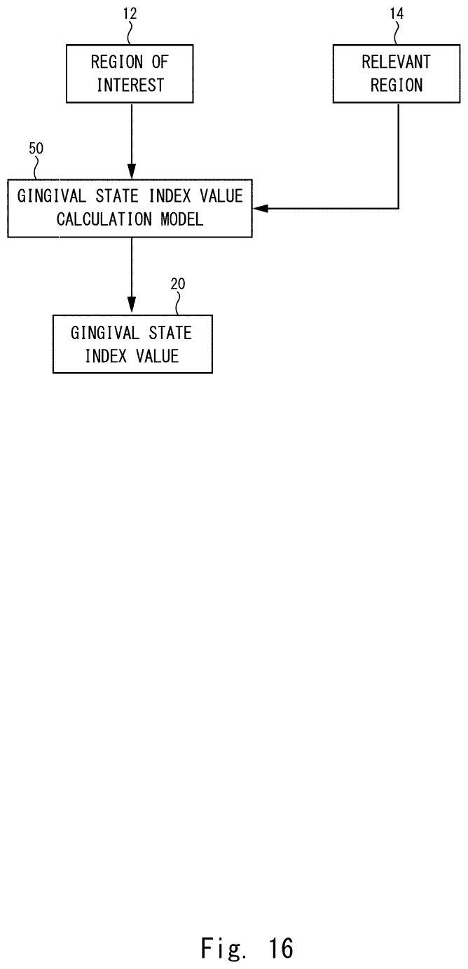

FIG. 16 is a diagram illustrating a case in which the gingival state index value 20 is calculated by the gingival state index value calculation model 50 into which the ROI 12 and the relevant region 14 are input. The calculation unit 2040 inputs both the ROI 12 and the relevant region 14 into the gingival state index value calculation model 50. The gingival state index value calculation model 50 outputs the gingival state index value 20 in response to the input of the ROI 12 and the relevant region 14.

Suppose that the gingival state index value 20 is calculated using the first prediction model 170. In this case, the first prediction model 170 is trained in advance to output a predicted value of a specific parameter in response to the input of both the ROI 12 and the relevant region 14.

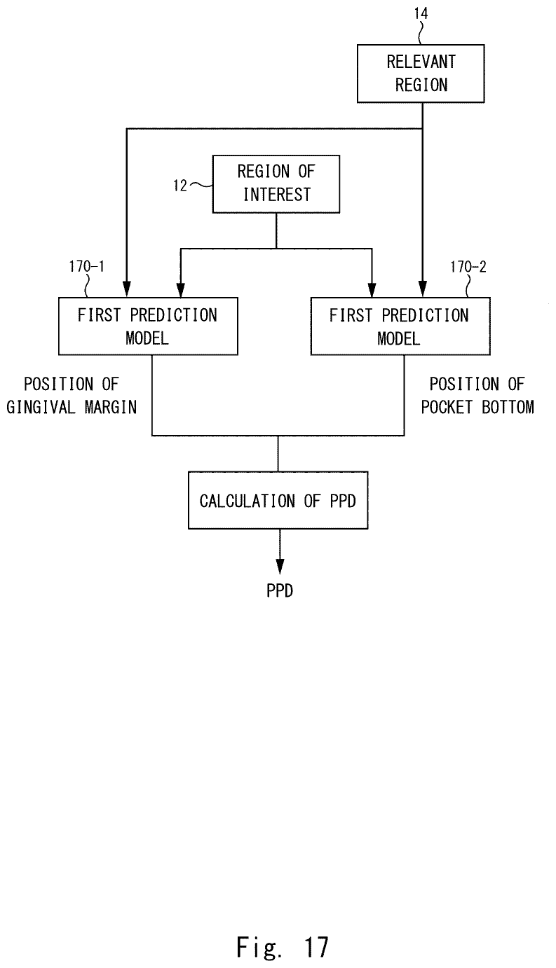

FIG. 17 is a diagram illustrating a case in which PPD is calculated using the first prediction model 170 into which the ROI 12 and the relevant region 14 are input. The first prediction model 170-1 is trained to output the position of the gingival margin in response to the input of the ROI 12 and the relevant region 14. The first prediction model 170-2 is trained to output the position of the pocket bottom in response to the input of the ROI 12 and the relevant region 14.

The calculation unit 2040 inputs both the ROI 12 and the relevant region 14 into the first prediction models 170-1 and 170-2. The first prediction model 170-1 outputs a predicted value of the position of the gingival margin in response to the input of the ROI 12 and the relevant region 14. The first prediction model 170-2 outputs a predicted value of the position of the pocket bottom in response to the input of the ROI 12 and the relevant region 14. The calculation unit 2040 calculates a difference between the position of the gingival margin obtained from the first prediction model 170-1 and the position of the pocket bottom obtained from the first prediction model 170-2, to calculate the PPD.

Into the gingival state index value calculation model 50 or the first prediction model 170, a feature value calculated from the relevant region 14 may be input in place of the relevant region 14. The feature value calculated from the relevant region 14 is referred to as a second feature value.

For the second feature value, various types of data that can be used as the first feature value as described above can be used. Furthermore, in a method of calculating the second feature value from the relevant region 14, the same method as the method of calculating the first feature value 100 from the ROI 12 can be used.

Here, suppose that a machine learning model is used for the process of calculating the second feature value from the relevant region 14. Hereinafter, a model that calculates the second feature value from the relevant region 14 is referred to as a second feature value calculation model. In this case, the first feature value calculation model 40 may be used as the second feature value calculation model. That is, in this case, data obtained by inputting the relevant region 14 into the first feature value calculation model 40 is handled as the second feature value.

The use of the relevant region 14 in addition to the ROI 12 in the process of calculating the gingival state index value 20 of the tooth of interest is advantageous in that the gingival state index value 20 for the tooth of interest can be calculated (predicted) with higher accuracy. Reasons for this will be described below.

In addition, periodontitis is more likely to develop locally than uniformly throughout the oral cavity. Therefore, even if the tooth of interest or the periodontium of interest develops periodontitis, another tooth or another periodontium may not have periodontitis. Therefore, comparison of the tooth of interest or the periodontium of interest with the other tooth or periodontium may make it possible to compare the tooth that has periodontitis or periodontium of the tooth with the tooth that does not have any periodontitis or periodontium of the tooth. Therefore, the gingival state index value 20 for the tooth of interest can be calculated with higher accuracy.

Similarly, even if a portion of the tooth of interest or a portion of the periodontium of interest has periodontitis, another portion of the tooth of interest or another portion of the periodontium of interest may not have periodontitis. Therefore, comparing of a portion of the tooth of interest or a portion of the periodontium of interest with another portion of the tooth of interest or another portion of the periodontium of interest may make it possible to compare a portion having periodontitis with a portion that does not have periodontitis in the tooth of interest or periodontium of the tooth. Therefore, the gingival state index value 20 for the tooth of interest can be calculated with higher accuracy.

Note that there are various methods of extracting the relevant region from the intraoral 3D data 10. For example, the calculation unit 2040 includes a machine learning model trained to extract the relevant region from the intraoral 3D data 10. Hereinafter, a model that extracts the relevant region from the intraoral 3D data 10 is referred to as a relevant region extraction model. As the relevant region extraction model, any machine learning model (for example, a neural network such as a CNN) capable of extracting a predetermined 3D region from 3D data can be used.

For example, the intraoral 3D data 10 and a position of the ROI are input into the relevant region extraction model. The relevant region extraction model is configured to output one or more relevant regions corresponding to the ROI located at the specified position from the intraoral 3D data 10.

The model may not be used in extracting the relevant region. For example, the calculation unit 2040 may analyze the intraoral 3D data 10 with the predetermined algorithm, to extract the relevant region from the intraoral 3D data 10.

In addition to or in place of the relevant region, various attribute information related to the subject person may be used to calculate the gingival state index value 20. The attribute information of the subject person includes race, age, gender, medical history, and pathology of the subject person (e.g., diabetes, periodontitis, gingivitis, endodontic lesion, root fracture, cementum detachment, caries, subgingival caries, or occlusal trauma), treatment history, smoking history, chief complaint, or the like.

By using information representing attributes of the subject person to calculate the gingival state index value 20, the gingival state index value 20 can be calculated (predicted) with higher accuracy in consideration of the attributes of the subject person.

In the case where the gingival state index value calculation model 50 is used to calculate the gingival state index value 20, the gingival state index value calculation model 50 is configured so that the attribute information of the subject person or the feature value calculated from the attribute information of the subject person is further input into the model. The gingival state index value calculation model 50 further uses the attribute information of the subject person or the feature value calculated from the attribute information of the subject person, to calculate the gingival state index value 20.

In the case where the first prediction model 170 is used to calculate the gingival state index value 20, the first prediction model 170 is configured so that the attribute information of the subject person or the feature value calculated from the attribute information of the subject person is further input into the model. The first prediction model 170 further uses the attribute information of the subject person or the feature value calculated from the attribute information of the subject person, to calculate a predicted value of a specific parameter.

The calculation unit 2040 may calculate the gingival state index value 20 using time series data of the ROI 12, the relevant region 14, or the attribute information of the subject person. A machine learning model capable of handling the time-series data, such as the RNN, can be used to calculate the gingival state index value 20 using the time-series data. By using the time-series data to calculate the gingival state index value 20, the gingival state index value 20 can be calculated (predicted) with higher accuracy in consideration of changes over time in subject person's teeth, periodontium, attributes, or the like.

### <<Method of Calculating Alveolar Bone Resorption Index Value 30>>

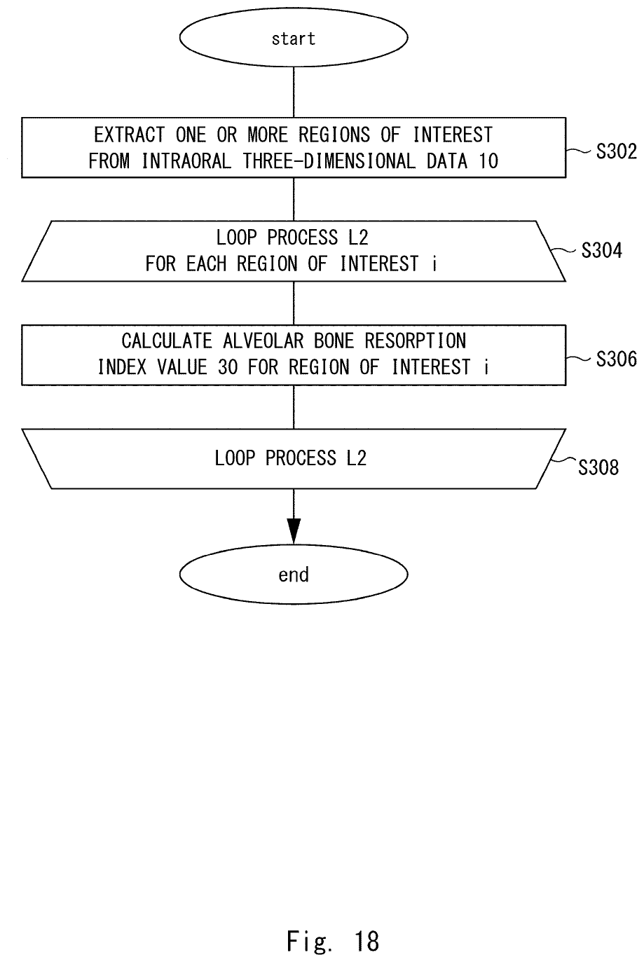

For example, the calculation unit 2040 extracts the ROI 12 from the intraoral 3D data 10 and calculates the alveolar bone resorption index value 30 using the ROI 12. FIG. 18 is a flowchart illustrating a flow of processes of calculating the alveolar bone resorption index value 30. The calculation unit 2040 extracts one or more ROIs 12 from the intraoral 3D data 10 (S302). Steps S304 to S308 constitute a loop process L2 executed for each ROI 12. In S304, the calculation unit 2040 determines whether the loop process L2 has been executed for all the ROIs 12. In a case where the loop process L2 has already been executed for all the ROIs 12, the processing of FIG. 18 ends.

In the case where there is one or more ROI 12 that is not yet subjected to the loop process L2, the calculation unit 2040 selects one of the ROIs 12 that are not yet subjected to the loop process L2. The ROI 12 selected here is denoted as an ROI i.

The calculation unit 2040 calculates the alveolar bone resorption index value 30 for the ROI i (S306). S308 is the end of the loop process L2, and hence S304 is executed again.

The process (S306) of calculating the alveolar bone resorption index value 30 from the ROI 12 is performed, for example, using a trained machine learning model. Hereinafter, a model that calculates the alveolar bone resorption index value 30 is referred to as an alveolar bone resorption index value calculation model.

For the alveolar bone resorption index value calculation model, for example, various models described above as the examples of the gingival state index value calculation model can be adopted. Furthermore, for the alveolar bone resorption index value calculation model, a model that combines various models and makes comprehensive determination can be adopted.

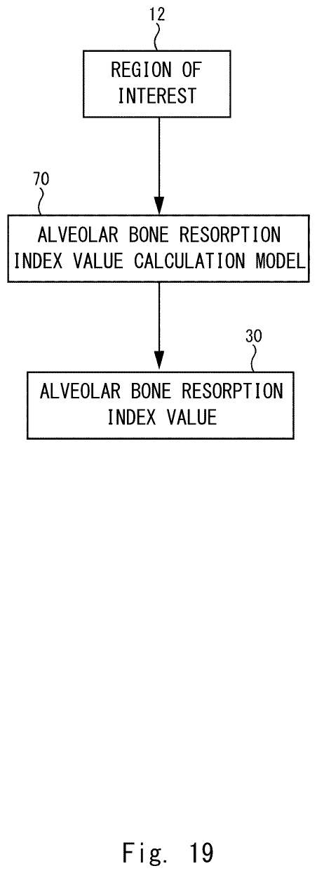

FIG. 19 is a diagram illustrating a case in which the alveolar bone resorption index value 30 is calculated using the alveolar bone resorption index value calculation model. In FIG. 19, an alveolar bone resorption index value calculation model 70 is trained in advance to output the alveolar bone resorption index value 30 in response to the input of the ROI 12.

The calculation unit 2040 inputs the ROI 12 extracted from the intraoral 3D data 10 into the alveolar bone resorption index value calculation model 70. By doing so, the calculation unit 2040 obtains the alveolar bone resorption index value 30 for the ROI 12 from the alveolar bone resorption index value calculation model 70.

Here, in the case where the calculation unit 2040 calculates a plurality of types of alveolar bone resorption index values 30, the calculation unit 2040 may include alveolar bone resorption index value calculation models 70 for respective types of alveolar bone resorption index values 30. Suppose that an alveolar bone resorption bone level and an alveolar bone resorption ratio are calculated as the alveolar bone resorption index values 30. In this case, the calculation unit 2040 includes an alveolar bone resorption index value calculation model 70 that is trained to calculate the alveolar bone resorption bone level and an alveolar bone resorption index value calculation model 70 that is trained to calculate the alveolar bone resorption ratio.

However, in the case where two index values (e.g., the alveolar bone resorption bone level and the alveolar bone resorption ratio) that are in a relationship in which one can be calculated from the other are handled, only one of the index values may be calculated using the model. For example, the calculation unit 2040 may calculate the alveolar bone resorption bone level by using the alveolar bone resorption index value calculation model 70 and may calculate the alveolar bone resorption ratio from the alveolar bone resorption bone level.

The calculation unit 2040 may include the alveolar bone resorption index value calculation model 70 for each tooth or tooth part. Here, suppose that the number of teeth is N. In the case where the alveolar bone resorption index value 30 is calculated for each tooth, the calculation unit 2040 includes N alveolar bone resorption index value calculation models 70 for each type of index value calculated as the alveolar bone resorption index value 30.

Furthermore, in the case where the index values for M parts of each tooth are calculated, the calculation unit 2040 includes M*N alveolar bone resorption index value calculation models 70 for each type of index value calculated as the alveolar bone resorption index value 30.

In consideration of the left-right symmetry via the oral midline as the reference, the number of alveolar bone resorption index value calculation models 70 may be set to half of the number described above. In this case, the same alveolar bone resorption index value calculation models 70 is used for two ROIs 12 that are located at left right symmetrical positions via the oral midline as the reference, respectively.

Thus, preparing the alveolar bone resorption index value calculation model 70 for each tooth or tooth part has an advantage that the alveolar bone resorption index value 30 can be calculated with higher accuracy than in a case of using the alveolar bone resorption index value calculation model 70 common to all teeth and tooth parts.

The alveolar bone resorption index value calculation model 70 may not be used in calculating the alveolar bone resorption index value 30. For example, the calculation unit 2040 may be configured to predict a value of a parameter that is required for calculating the alveolar bone resorption index value 30 using a machine learning model. Hereinafter, a model that predicts the value of the parameter required for calculating the alveolar bone resorption index value 30 is referred to as a second prediction model. A type of machine learning model that can be used as the second prediction model is the same as the type of machine learning model that can be used as the first prediction model.

The second prediction model is trained in advance to output a value of a specific parameter in response to the input of the ROI 12. The calculation unit 2040 inputs the ROI 12 into the second prediction model and calculates the alveolar bone resorption index value 30 by using the value of the parameter obtained from the second prediction model.

The parameter required for calculating the alveolar bone resorption index value 30 varies with a type of index value calculated as the alveolar bone resorption index value 30. Suppose that the alveolar bone resorption bone level is calculated as the alveolar bone resorption index value 30. The alveolar bone resorption bone level is calculated as B/A, wherein A is a distance from the CEJ to the root apex and B is a distance from the CEJ to the alveolar crest. Accordingly, it is necessary for calculating the alveolar bone resorption bone level to determine a position of the CEJ, a position of the root apex, and a position of the alveolar crest.

For example, the calculation unit 2040 includes a second prediction model that is trained to predict the position of the CEJ for the ROI 12, a second prediction model that is trained to predict the position of the root apex for the ROI 12, and a second prediction model trained to predict the alveolar crest for the ROI 12. The calculation unit 2040 calculates the alveolar bone resorption bone level by using the second prediction models.

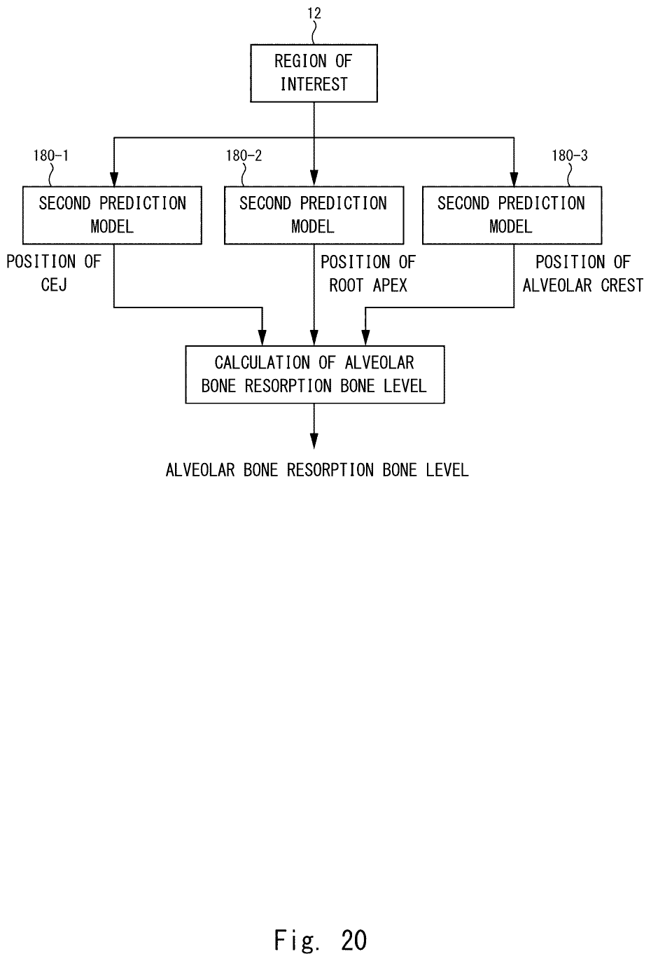

FIG. 20 is a diagram illustrating a case in which the alveolar bone resorption bone level is calculated using the second prediction model. A second prediction model 180-1 is a second prediction model that is trained to predict the position of the CEJ in response to the input of the ROI 12. A second prediction model 180-2 is a second prediction model that is trained to predict the position of the root apex in response to the input of the ROI 12. A second prediction model 180-3 is a second prediction model that is trained to predict the alveolar crest in response to the input of the ROI 12.

The calculation unit 2040 inputs the ROI 12 into the second prediction model 180-1 to obtain the position of the CEJ. Furthermore, the calculation unit 2040 inputs the ROI 12 into the second prediction model 180-2 to obtain the position of the root apex. Furthermore, the calculation unit 2040 inputs the ROI 12 into the second prediction model 180-3 to obtain the position of the alveolar crest. The calculation unit 2040 then calculates the alveolar bone resorption bone level by using the position of the CEJ obtained from the second prediction model 180-1, the position of the tooth apex obtained from the second prediction model 180-2, and the position of the alveolar crest obtained from the second prediction model 180-3.

The alveolar bone resorption ratio can be calculated by multiplying the alveolar bone resorption bone level by 100. Therefore, also in the case where the alveolar bone resorption ratio is calculated as the alveolar bone resorption index value 30, the second prediction models 180-1 to 180-3 shown in FIG. 20 can be used.

Here, both the root apex and the alveolar crest are hidden by the gingiva (see FIG. 2). The calculation unit 2040 can predict the positions of the root apex and alveolar crest hidden by the gingiva by using second prediction models 180. In addition, the CEJ may be hidden by the gingiva. By using the second prediction model 180, the calculation unit 2040 can predict the position of the CEJ even if the CEJ is hidden by the gingiva. Therefore, the use of the second prediction model 180 can facilitate calculation of an index value that requires the position of a part hidden by the gingiva.

The calculation unit 2040 includes the second prediction model 180, for example, for each parameter used to calculate the alveolar bone resorption index value 30. For example, in the case where the alveolar bone resorption bone level and the alveolar bone resorption ratio are calculated as the alveolar bone resorption index values 30, the calculation unit 2040 includes the second prediction model 180 that predicts the position of the CEJ, the second prediction model 180 that predicts the position of the root apex, and the second prediction model 180 that predicts the position of the alveolar crest.

Here, there is a parameter that can be used in both the calculation of the gingival state index value 20 and the calculation of the alveolar bone resorption index value 30, such as the position of the CEJ. For this kind of parameter, the calculation unit 2040 may use a value output from the first prediction model 170 for both the calculation of the gingival state index value 20 and the calculation of the alveolar bone resorption index value 30.

Suppose that the calculation unit 2040 calculates the CAL as the gingival state index value 20 and calculates the alveolar bone resorption bone level as the alveolar bone resorption index value 30. In this case, the calculation unit 2040 inputs the ROI 12 into the first prediction model 170 that predicts the position of the CEJ and uses the value output from the first prediction model 170 in both the calculation of the CAL and the calculation of the alveolar bone resorption bone level. Therefore, in this case, the calculation unit 2040 does not have to include the second prediction model 180 that predicts the position of the CEJ.

The second prediction model 180 may be prepared for each tooth or each tooth part. For example, in the case where the number of teeth is N, the calculation unit 2040 includes N second prediction models 180 for each parameter. Furthermore, in the case where the index value is calculated for M parts of each tooth, the calculation unit 2040 includes M*N second prediction models 180 for each parameter.

In consideration of the left-right symmetry via the oral midline as the reference, the number of alveolar bone resorption index value calculation models 70 may be set to half of the number described above. In this case, the same second prediction model 180 is used for two ROIs 12 that are located at left and right symmetrical positions via the oral midline as the reference, respectively.

Thus, preparing the second prediction model 180 for each tooth or tooth part has an advantage that the value of the parameter can be predicted with higher accuracy than in a case of using the second prediction model 180 common to all teeth and tooth parts.

The data input into the alveolar bone resorption index value calculation model 70 and the second prediction model 180 may be a feature value that can be calculated from the ROI 12, rather than the ROI 12. Hereinafter, the feature value calculated from the ROI 12 and used to calculate the alveolar bone resorption index value 30 is referred to as a third feature value.

The third feature value is a value representing a predetermined type of feature related to the tooth of interest, or a value representing a predetermined type of feature related to the periodontium of interest in the same manner as in, for example, the first feature value. However, the type of feature used to calculate the gingival state index value 20 and the type of feature used to calculate the alveolar bone resorption index value 30 may differ from each other.

In a method of calculating the third feature value from the ROI 12, the same method as the method of calculating the first feature value from the ROI 12 can be used. For example, the third feature value is calculated using a machine learning model trained in advance. Hereinafter, a model that is used for calculating the third feature value is referred to as a third feature value calculation model. A type of model that can be used as the third feature value calculation model is the same as the type of model that can be used as the first feature value calculation model.

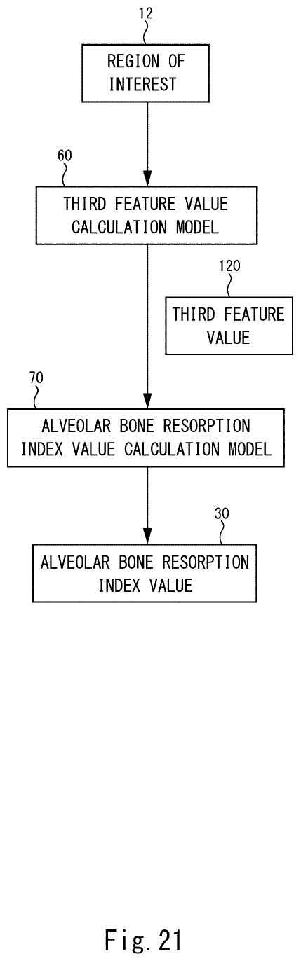

FIG. 21 is a diagram illustrating a case in which the alveolar bone resorption index value 30 is calculated using the third feature value calculation model and the alveolar bone resorption index value calculation model 70. In FIG. 21, a third feature value calculation model 60 is trained in advance to output a third feature value 120 in response to the input of the ROI 12. Furthermore, the alveolar bone resorption index value calculation model 70 is trained in advance to output the alveolar bone resorption index value 30 in response to the input of the third feature value 120.

The calculation unit 2040 inputs the ROI 12 extracted from the intraoral 3D data 10 into the third feature value calculation model 60. Furthermore, the calculation unit 2040 inputs the third feature value 120 output from the third feature value calculation model 60 into the alveolar bone resorption index value calculation model 70. By doing so, the calculation unit 2040 obtains the alveolar bone resorption index value 30 for the tooth of interest included in the ROI 12 from the alveolar bone resorption index value calculation model 70.

Here, in the case where the calculation unit 2040 includes a plurality of alveolar bone resorption index value calculation models 70, the third feature value calculation model 60 may be shared by all the alveolar bone resorption index value calculation models 70, or the third feature value calculation model 60 may be prepared for each alveolar bone resorption index value calculation model 70.

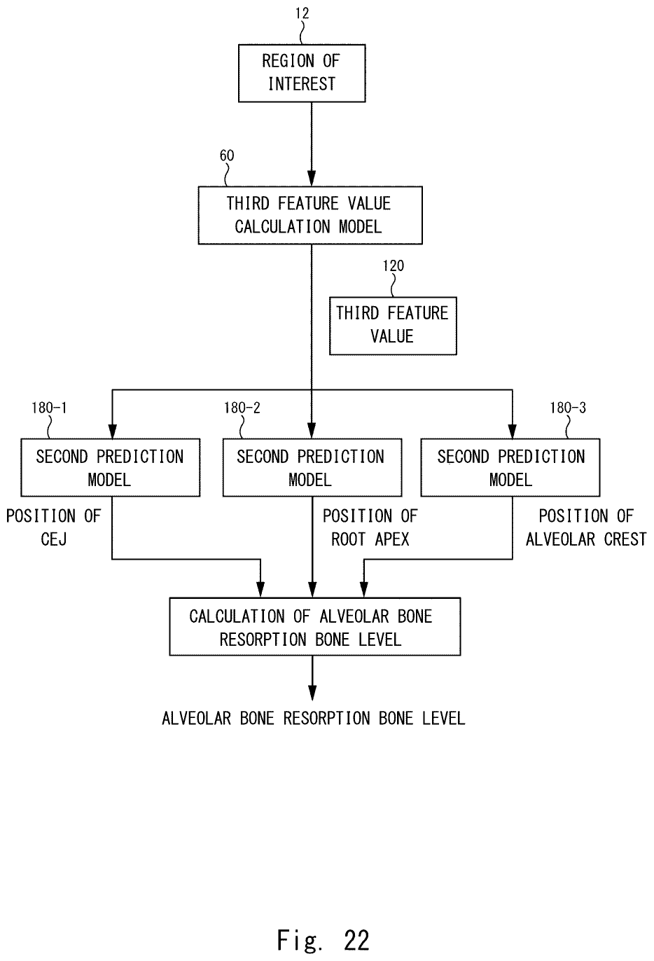

FIG. 22 is a diagram illustrating a case in which the alveolar bone resorption bone level is calculated using the third feature value calculation model 60 and the second prediction model 180. The second prediction model 180-1 is trained in advance to output the position of the CEJ in response to the input of the third feature value 120. The second prediction model 180-2 is trained in advance to output the position of the root apex in response to the input of the third feature value 120. The second prediction model 180-3 is trained in advance to output the position of the alveolar crest in response to the input of the third feature value 120.

The calculation unit 2040 inputs the ROI 12 extracted from the intraoral 3D data 10 into the third feature value calculation model 60. Furthermore, the calculation unit 2040 inputs the third feature value 120 output from the third feature value calculation model 60 into each of the second prediction models 180-1, 180-2, and 180-3. The calculation unit 2040 then calculates differences in the position of the CEJ obtained from the second prediction model 180-1 and the position of the root apex obtained from the second prediction model 180-2 and the position of the alveolar crest obtained from the second prediction model 180-3, to calculate the alveolar bone resorption bone level.

Here, in the case where the calculation unit 2040 includes a plurality of second prediction models 180, the third feature value calculation model 60 may be shared by all the second prediction models 180, or the third feature value calculation model 60 may be prepared for each second prediction model 180.

As the third feature value 120, the first feature value 100 may be used. In this case, in the process of calculating the gingival state index value 20 (S206) and the process of calculating the alveolar bone resorption index value 30 (S306), the same feature value is used. In this case, the calculation unit 2040 may not include the third feature value calculation model 60.

The third feature value 120 may be calculated without using the machine learning model. In this case, the calculation unit 2040 analyzes the ROI 12 with the predetermined algorithm, to calculate a value representing a predetermined type of feature related to the tooth of interest or a predetermined type of feature related to the periodontium of the tooth of interest as the third feature value 120.

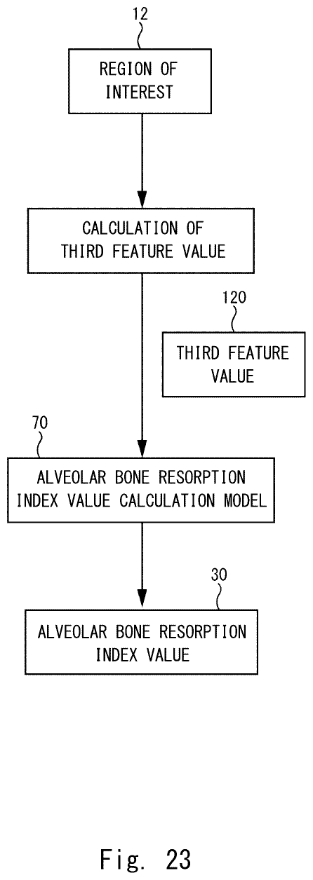

FIG. 23 is a diagram illustrating a case in which the third feature value calculated without using the third feature value calculation model 60 is used by the alveolar bone resorption index value calculation model 70. The calculation unit 2040 analyzes the ROI 12 to calculate the third feature value 120.

Thereafter, the calculation unit 2040 inputs the calculated third feature value 120 into the alveolar bone resorption index value calculation model 70. By doing so, the alveolar bone resorption index value 30 is obtained from the alveolar bone resorption index value calculation model 70.

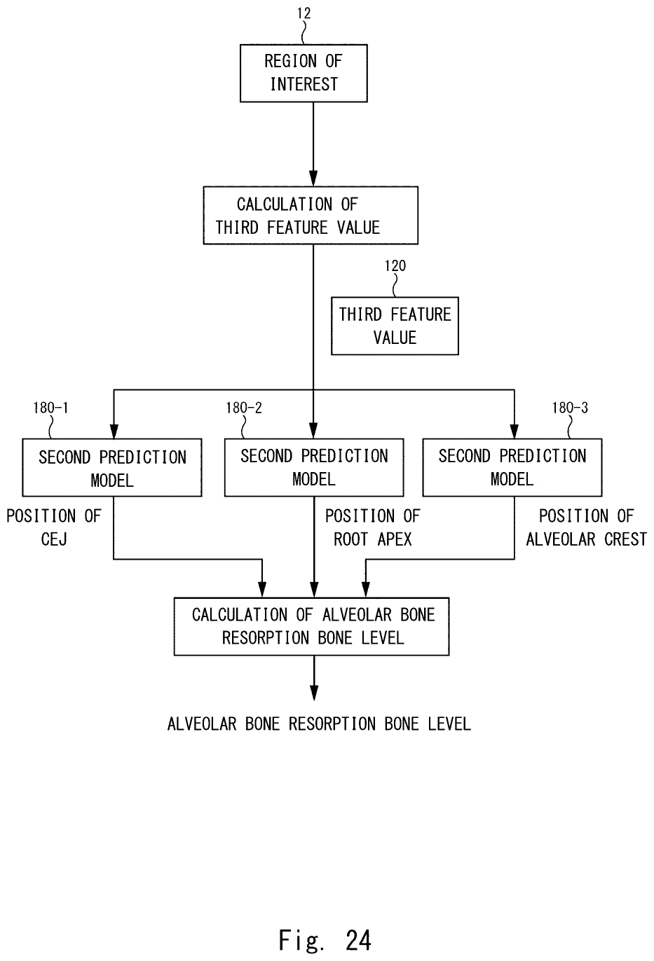

FIG. 24 is a diagram illustrating a case in which the third feature value calculated without using the third feature value calculation model 60 is used by the second prediction model 180. In the example of FIG. 24, the alveolar bone resorption bone level is calculated as the alveolar bone resorption index value 30.

The calculation unit 2040 analyzes the ROI 12 to calculate the third feature value 120. Thereafter, the calculation unit 2040 inputs the calculated third feature value 120 into each of the second prediction models 180-1, 180-2, and 180-3. The calculation unit 2040 then calculates the alveolar bone resorption bone level based on the position of the CEJ obtained from the second prediction model 180-1, the position of the root apex obtained from the second prediction model 180-2, and the position of the alveolar crest obtained from the second prediction model 180-3.

In the case where the alveolar bone resorption index value 30 is calculated from the ROI 12, the relevant region 14 described above may be further used. Suppose that the alveolar bone resorption index value 30 is calculated using the alveolar bone resorption index value calculation model 70. In this case, the alveolar bone resorption index value calculation model 70 is trained in advance to output the alveolar bone resorption index value 30 in response to the input of both the ROI 12 and the relevant region 14.

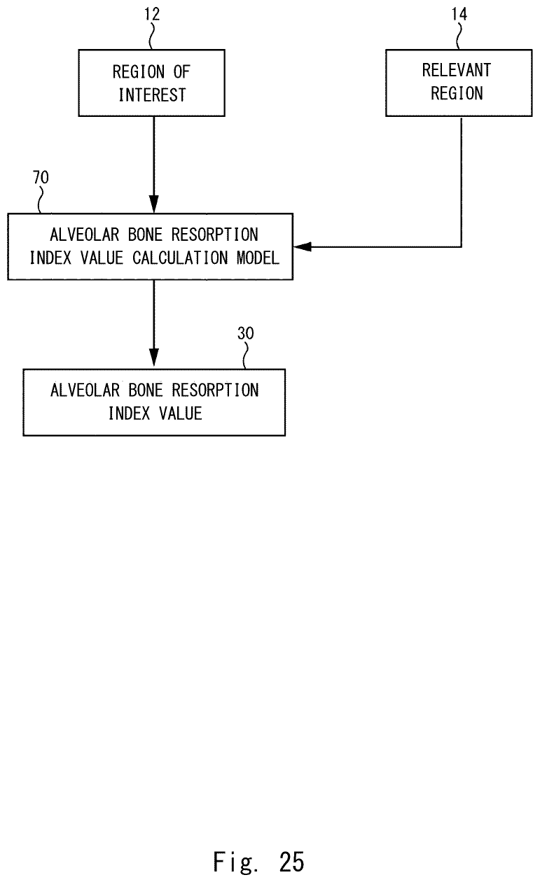

FIG. 25 is a diagram illustrating a case in which the alveolar bone resorption index value 30 is calculated by the alveolar bone resorption index value calculation model 70 into which the ROI 12 and the relevant region 14 are input. The calculation unit 2040 inputs both the ROI 12 and the relevant region 14 into the alveolar bone resorption index value calculation model 70. The alveolar bone resorption index value calculation model 70 outputs the alveolar bone resorption index value 30 in response to the input of the ROI 12 and the relevant region 14.

Suppose that the alveolar bone resorption index value 30 is calculated using the second prediction model 180. In this case, the second prediction model 180 is trained in advance to output a predicted value of a specific parameter in response to the input of both the ROI 12 and the relevant region 14.

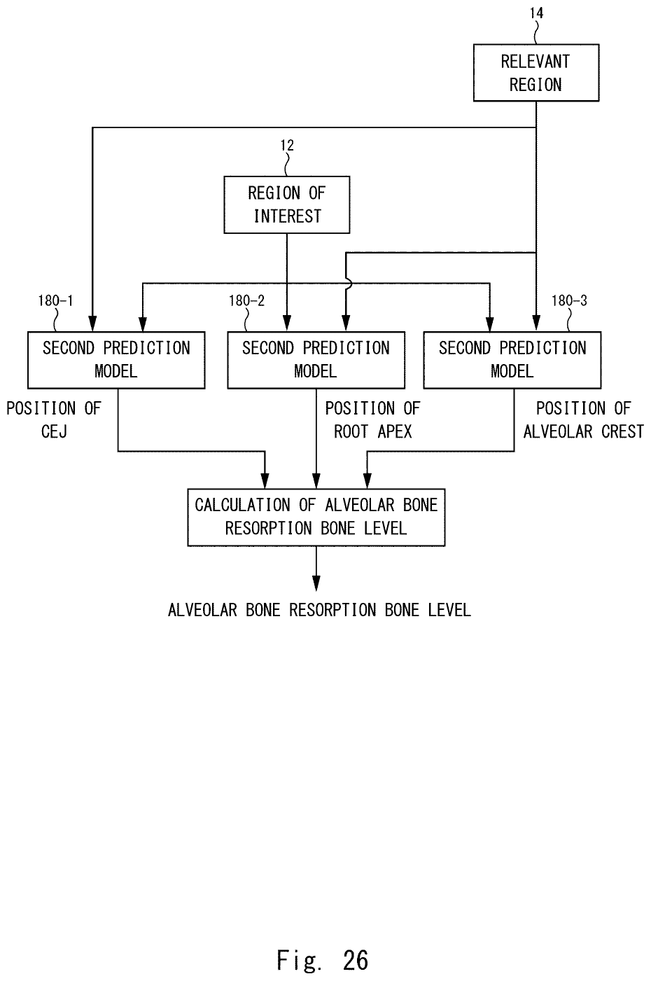

FIG. 26 is a diagram illustrating a case in which the alveolar bone resorption bone level is calculated using the second prediction model 180 into which the ROI 12 and the relevant region 14 are input. The second prediction model 180-1 is trained in advance to output the position of the CEJ in response to the input of the ROI 12 and the relevant region 14. The second prediction model 180-2 is trained in advance to output the position of the root apex in response to the input of the ROI 12 and the relevant region 14. The second prediction model 180-3 is trained in advance to output the position of the alveolar crest in response to the input of the ROI 12 and the relevant region 14.

The calculation unit 2040 inputs both the ROI 12 and the relevant region 14 into each of the second prediction models 180-1, 180-2, and 180-3. The second prediction model 180-1 outputs a predicted value of the position of the CEJ in response to the input of the alveolar bone resorption index value 30 and the relevant region 14. The second prediction model 180-2 outputs a predicted value of the position of the root apex in response to the input of the alveolar bone resorption index value 30 and the relevant region 14. The second prediction model 180-3 outputs a predicted value of the position of the alveolar crest in response to the input of the alveolar bone resorption index value 30 and the relevant region 14. The calculation unit 2040 calculates the alveolar bone resorption bone level by using the position of the CEJ obtained from the second prediction model 180-1, the position of the apical apex obtained from the second prediction model 180-2, and the position of the alveolar crest obtained from the second prediction model 180-3.

Into the alveolar bone resorption index value calculation model 70 and the second prediction model 180, a feature value calculated from the relevant region 14 may be input in place of the relevant region 14. The feature value that is calculated from the relevant region 14 and used to calculate the alveolar bone resorption index value 30 is referred to as a fourth feature value.

For the fourth feature value, various types of data that can be used as the first feature value described above can be used. Furthermore, in a method of calculating the fourth feature value from the relevant region 14, the same method as the method of calculating the first feature value 100 from the ROI 12 can be used.

Here, suppose that a machine learning model is used in the process of calculating the fourth feature value from the relevant region 14. Hereinafter, a model that calculates the fourth feature value from the relevant region 14 is referred to as a fourth feature value calculation model. In this case, the third feature value calculation model 60 may be used as the fourth feature value calculation model. That is, in this case, data obtained by inputting the relevant region 14 into the third feature value calculation model 60 is handled as the fourth feature value.

The use of the relevant region 14, in addition to the ROI 12, in the process of calculating the alveolar bone resorption index value 30 of the tooth of interest has an advantage that the alveolar bone resorption index value 30 for the tooth of interest can be calculated (predicted) with higher accuracy. A reason for this is the same as the reason the gingival state index value 20 can be calculated with higher accuracy for the tooth of interest by using the relevant region 14 in addition to the ROI 12 in the processing of calculating the gingival state index value 20 of the tooth of interest.

In addition to or in place of the relevant region, various attribute information related to the subject person may be used to calculate the alveolar bone resorption index value 30. The type of attribute information of the subject person is as described above. By using information representing the attributes of the subject person in the calculation of the alveolar bone resorption index value 30, the alveolar bone resorption index value 30 can be calculated (predicted) with higher accuracy in consideration of the attributes of the subject person.

In the case where the alveolar bone resorption index value calculation model 70 is used to calculate the alveolar bone resorption index value 30, the alveolar bone resorption index value calculation model 70 is configured so that the attribute information of the subject person or a feature value calculated from the attribute information of the subject person is further input into the model. The alveolar bone resorption index value calculation model 70 further uses the attribute information of the subject person or the feature value calculated from the attribute information of the subject person, to calculate the alveolar bone resorption index value 30.

In the case where the second prediction model 180 is used to calculate the alveolar bone resorption index value 30, the second prediction model 180 is configured so that the attribute information of the subject person or the feature value calculated from the attribute information of the subject person is further input into the model. The second prediction model 180 further uses the attribute information of the subject person or the feature value calculated from the attribute information of the subject person, to calculate a predicted value of a specific parameter.

The calculation unit 2040 may calculate the alveolar bone resorption index value 30 by using time series data of the ROI 12, the relevant region 14, or the attribute information of the subject person. A machine learning model capable of handling the time-series data, such as RNN, can be used to calculate the alveolar bone resorption index value 30 using the time-series data. By using the time-series data in calculating the alveolar bone resorption index value 30, the alveolar bone resorption index value 30 can be calculated (predicted) with higher accuracy in consideration of changes over time in the subject person's teeth, periodontium, attributes, or the like.

### <Output of Result>

The index value calculation apparatus 2000 can output the gingival state index value 20, the alveolar bone resorption index value 30, or both of them calculated by the calculation unit 2040 in any manner. Hereinafter, information output by the index value calculation apparatus 2000 is referred to as output information.

The output information indicates an index value calculated by the index value calculation apparatus 2000. Here, in the case where the index value is calculated for each of a plurality of teeth included in the intraoral 3D data 10, it is preferable that the output information indicates the index value calculated for the tooth together with information capable of identifying the tooth. For example, the index value calculation apparatus 2000 assigns an identification number to each of the plurality of teeth included in the intraoral 3D data 10 according to a predetermined rule. The index value calculation apparatus 2000 generates the output information that indicates the identification number of each tooth in association with the index value calculated for the tooth.

Here, in a case where a plurality of ROIs is extracted for one tooth, a plurality of index values is calculated for the one tooth. Therefore, in this case, the index value calculation apparatus 2000 assigns an identification number to each part for which the index value is calculated.

Any rule can be used in the predetermined rule for assigning the identification number to the tooth or tooth part. For example, an identification number for use by those skilled in the art can be used.

The output information can be output in various manners. For example, the index value calculation apparatus 2000 stores the output information in an arbitrary storage unit. Furthermore, for example, the index value calculation apparatus 2000 displays the output information on a display device. In another example, the index value calculation apparatus 2000 transmits the output information to another apparatus (for example, a user terminal).

### <Way of Using Index Value>

The gingival state index value 20 and the alveolar bone resorption index value 30 can be used in various ways. For example, the gingival state index value 20 and the alveolar bone resorption index value 30 can be used to determine presence or absence of a periodontal-related disease (e.g., gingivitis), to determine a state of the periodontal-related disease, or to determine whether to recommend a visit to a dentist. These determinations may be performed manually or by using a computer. In the latter case, the determination process may be performed by the index value calculation apparatus 2000 or by an apparatus other than the index value calculation apparatus 2000.

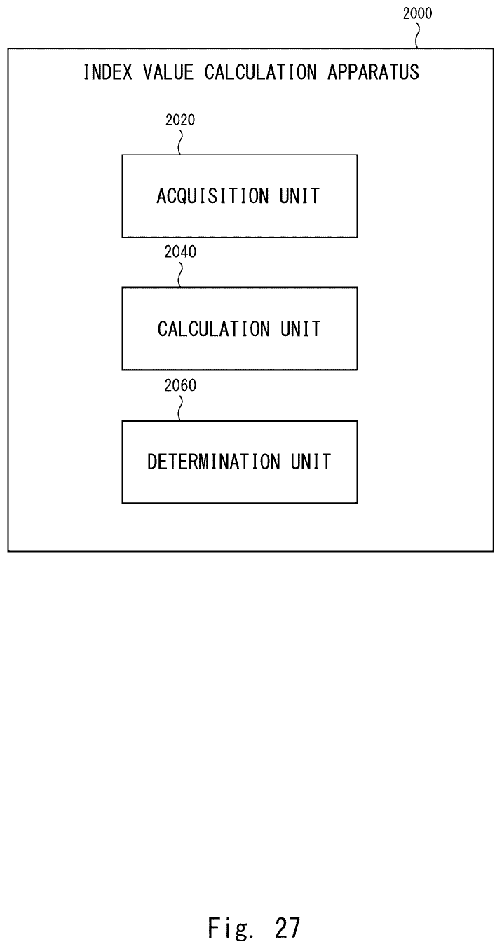

FIG. 27 is a block diagram illustrating a functional configuration of the index value calculation apparatus 2000 that performs the determination process with the index value. In FIG. 27, the index value calculation apparatus 2000 further includes a determination unit 2060. The determination unit 2060 performs the determination process using the gingival state index value 20, the alveolar bone resorption index value 30, or both of them calculated by the calculation unit 2040. The determination process is, for example, one or more of the process of determining the presence or absence of the periodontal-related disease, the process of determining the state of the periodontal-related disease, and the process of determining whether to recommend the visit to the dentist described above.

The determination process by the determination unit 2060 using the gingival state index value 20 and the alveolar bone resorption index value 30 can be implemented, for example, in accordance with guidelines widely recognized and used by those skilled in the art, such as Guidelines for Periodontal Treatment 2022 of the Japanese Society of Periodontology, 2018 New Classification of Periodontal Disease of the American Academy of Periodontology/European Federation to Periodontology, the community periodontal index classification by World Health Organization (WHO), the periodontal disease examination manual by the Ministry of Health, Labor and Welfare, or the like.

The index value calculation apparatus 2000 may have still other functions. For example, the index value calculation apparatus 2000 may have a function of calculating a gingival index (GI) from the intraoral 3D data 10 and a function of predicting a plaque control state. Furthermore, the index value calculation apparatus 2000 may have a function of diagnosing and predicting a caries status of teeth, prosthetic status of teeth, and status of implanted teeth from the intraoral 3D data 10. In addition, the index value calculation apparatus 2000 may further have a function of introducing a dentist, making a dentist appointment, or the like, in response to the determination unit 2060 determining to recommend the visit to the dentist.

### <Regarding Training of Model>

As described above, one or more models may be used in the index value calculation apparatus 2000. Each model is trained in advance before used by the index value calculation apparatus 2000.

An apparatus for use in training the model is called a training apparatus. The training apparatus may be the index value calculation apparatus 2000 or an apparatus other than the index value calculation apparatus 2000.

The training apparatus generates a model with initial values set to trainable parameters (in other words, generates and initializes the model). The training apparatus then trains the model by repeatedly updating the trainable parameters of the model with a plurality of training data. For example, if the model is a neural network, the trainable parameters include a weight given to each edge and biases.

Hereinafter, a method of training each model will be described in more detail.

### <<Training of Model for Use in Calculation of Gingival State Index Value 20>>

For the calculation of the gingival state index value 20, the gingival state index value calculation model 50 and the first prediction model 170 may be used. For the use of the gingival state index value calculation model 50, the training apparatus trains the gingival state index value calculation model 50.

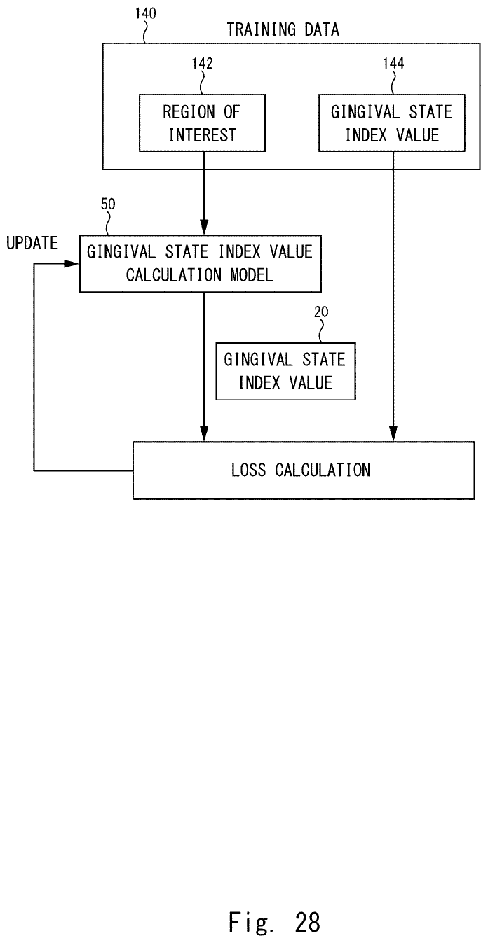

FIG. 28 is a diagram illustrating the training of the gingival state index value calculation model 50. Training data 140 includes an ROI 142 and a gingival state index value 144. The ROI 142 is a 3D region including a tooth of interest and periodontium of the tooth of interest in the same manner as in the ROI 12. The gingival state index value 144 is a ground truth gingival state index value to be calculated from the ROI 142 by the gingival state index value calculation model 50. The gingival state index value 144 may be calculated, for the tooth of interest and periodontium of the tooth that are included in the ROI 142, by performing actual measurement using a probe or by performing actual measurement using the probe and an X-ray image.

The training apparatus inputs the ROI 142 into the gingival state index value calculation model 50 to obtain the gingival state index value 20. The training apparatus calculates a loss based on the gingival state index values 20 and 144, and updates each parameter of the gingival state index value calculation model 50 based on the calculated loss.

The training apparatus repeatedly updates the gingival state index value calculation model 50 with the plurality of training data 140. Thus, the training apparatus obtains the trained gingival state index value calculation model 50. The trained gingival state index value calculation model 50 is then used by the index value calculation apparatus 2000.

As described above, the index value calculation apparatus 2000 may include the gingival state index value calculation model 50 for each tooth or tooth part. In this case, the training apparatus trains each gingival state index value calculation model 50 prepared for each tooth or tooth part.

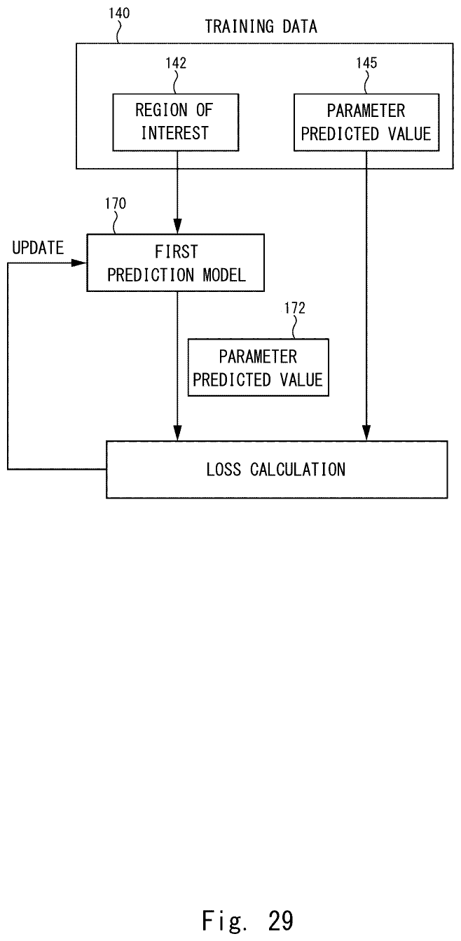

When the first prediction model 170 is used to calculate the gingival state index value 20, the training apparatus trains the first prediction model 170. FIG. 29 is a diagram illustrating the training of the first prediction model 170. The training data 140 includes the ROI 142 and a parameter predicted value 145. The parameter predicted value 145 indicates a ground truth predicted value to be output by the first prediction model 170. Suppose that the first prediction model 170 is a model that predicts the position of the pocket bottom. In this case, the parameter predicted value 145 indicates an actual measured value of the position of the pocket bottom in the ROI 142.

The training apparatus inputs the ROI 142 into the first prediction model 170 to obtain a parameter predicted value 172. The training apparatus calculates a loss based on the parameter predicted values 172 and 145, and updates each parameter of the first prediction model 170 based on the calculated loss.

The training apparatus repeatedly updates the first prediction model 170 using the plurality of training data 140. Thus, the training apparatus obtains the first prediction model 170 that has been trained. The trained first prediction model 170 is then used by the index value calculation apparatus 2000.

As described above, a plurality of parameters such as the position of the gingival margin, the position of the pocket bottom, and the position of the CEJ can be used to calculate the gingival state index value 20. Therefore, for each parameter, the training apparatus trains the first prediction model 170 used to predict the value of that parameter. For example, the first prediction model 170 that predicts the position of the gingival margin is trained using training data 140 indicating the actual measured value of the position of the gingival margin as the parameter predicted value 145. Furthermore, the first prediction model 170 that predicts the position of the pocket bottom is trained using the training data 140 indicating the actual measured value of the position of the pocket bottom as the parameter predicted value 145.

Furthermore, the index value calculation apparatus 2000 may include, for each tooth or tooth part, the first prediction model 170 for the same parameter. In this case, the training apparatus trains each first prediction model 170 prepared for each tooth or tooth part.

The first feature value calculation model 40 may be used to calculate the gingival state index value 20. In this case, the training apparatus also trains the first feature value calculation model 40. The training of the first feature value calculation model 40 may be performed together with the training of the gingival state index value calculation model 50 and the first prediction model 170, or may be performed independently of the training of the gingival state index value calculation model 50 and the first prediction model 170.

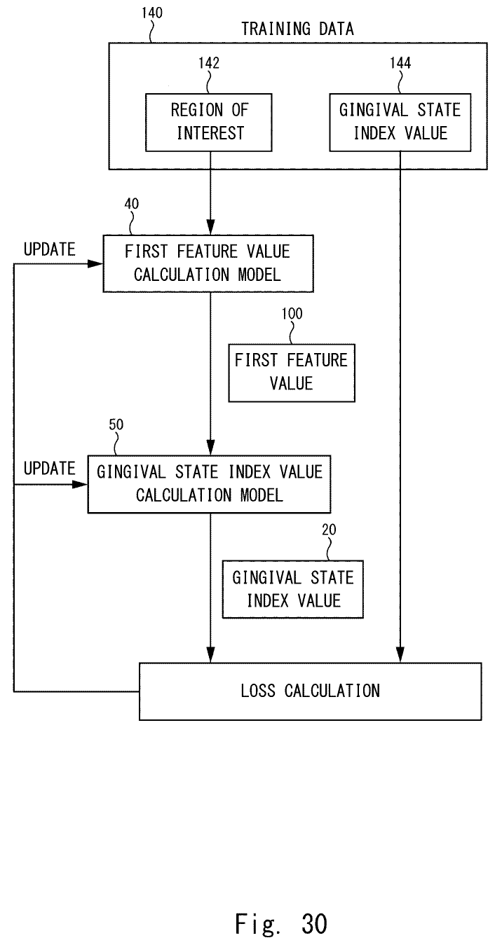

FIG. 30 is a diagram illustrating the training of the first feature value calculation model 40 and the gingival state index value calculation model 50. The training apparatus inputs the ROI 142 into the first feature value calculation model 40 to obtain the first feature value 100. Furthermore, the training apparatus inputs the first feature value 100 output from the first feature value calculation model 40 into the gingival state index value calculation model 50 to obtain the gingival state index value 20. The training apparatus calculates a loss based on the gingival state index values 20 and 144, and updates each parameter of the first feature value calculation model 40 and the gingival state index value calculation model 50 based on the calculated loss.

The training apparatus repeatedly updates the first feature value calculation model 40 and the gingival state index value calculation model 50 with the plurality of training data 140. Thus, the training apparatus obtains the first feature value calculation model 40 and gingival status index value calculation model 50 that have been trained. The trained first feature value calculation model 40 and the trained gingival state index value calculation model 50 are then used by the index value calculation apparatus 2000.

A method of training the first feature value calculation model 40 and the first prediction model 170 together is the same as the method of training the first feature value calculation model 40 and the gingival state index value calculation model 50 together. That is, the training apparatus inputs the ROI 142 into the first feature value calculation model 40 to obtain the first feature value 100. Furthermore, the training apparatus inputs the first feature value 100 output from the first feature value calculation model 40 into the first prediction model 170 to obtain the parameter predicted value 172. The training apparatus calculates a loss based on the parameter predicted values 172 and 145, and updates each parameter of the first feature value calculation model 40 and first prediction model 170 based on the calculated loss.

In the case where the first feature value calculation model 40 is trained independently, training data including the ROI and ground truth first feature value is used. The training apparatus calculates a loss based on the first feature value 100 obtained by inputting the ROI indicated in the training data into the first feature value calculation model 40 and the ground truth first feature value indicated in the training data. The training apparatus then updates each parameter of the first feature value calculation model 40 based on the calculated loss.

As described above, the first feature value calculation model 40 may not be used in calculating the first feature value 100. In this case, the first feature value 100 calculated from the ROI 142 with a predetermined algorithm is used to train the gingival state index value calculation model 50 and the first prediction model 170.

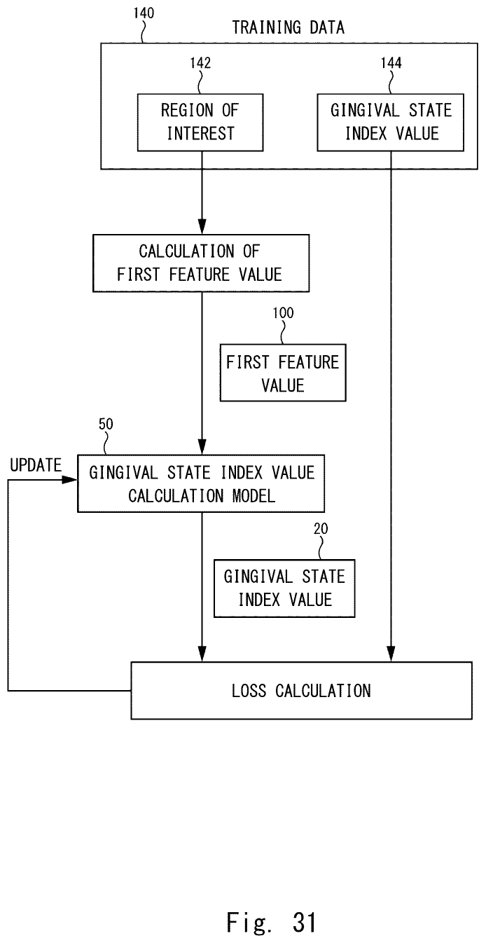

FIG. 31 is a diagram illustrating the training of the gingival state index value calculation model 50 into which the first feature value 100 calculated with a predetermined algorithm is input. The training apparatus analyzes the ROI 142 with the predetermined algorithm to calculate the first feature value 100. The training apparatus inputs the calculated first feature value 100 into the gingival state index value calculation model 50 to obtain the gingival state index value 20. The training apparatus calculates a loss based on the gingival state index values 20 and 144, and updates each parameter of the gingival state index value calculation model 50 based on the calculated loss.

The first prediction model 170 can be trained in the same manner. Specifically, the training apparatus analyzes the ROI 142 with the predetermined algorithm to calculate the first feature value 100, and inputs the first feature value 100 into the first prediction model 170. The training apparatus calculates a loss based on the parameter predicted value 172 output from the first prediction model 170 and the parameter predicted value 145, and updates each parameter of the first prediction model 170 based on the calculated loss.

In the case where the relevant region 14 is used to calculate the gingival state index value 20, the training data 140 including the relevant region is used to train the gingival state index value calculation model 50 and the first prediction model 170.

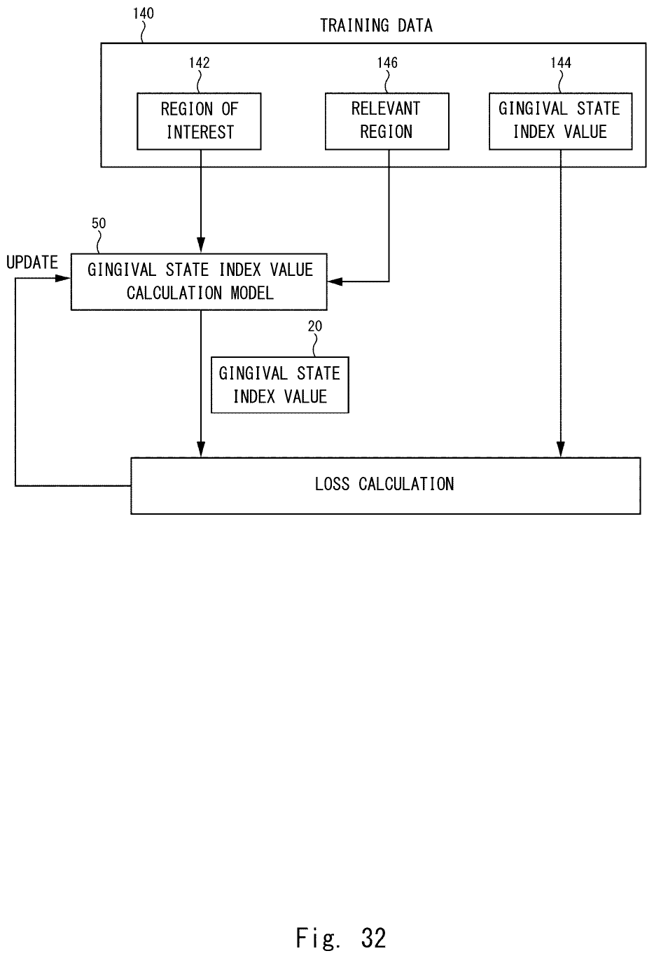

FIG. 32 is a diagram illustrating the training of the gingival state index value calculation model 50 using the relevant region. The training data 140 includes the ROI 142, a relevant region 146, and the gingival state index value 144. The relevant region 146 is a relevant region corresponding to the ROI 142.

The training apparatus inputs the ROI 142 and the relevant region 146 into the gingival state index value calculation model 50 to obtain the gingival state index value 20. The training apparatus calculates a loss based on the gingival state index values 20 and 144, and updates each parameter of the gingival state index value calculation model 50 based on the calculated loss.

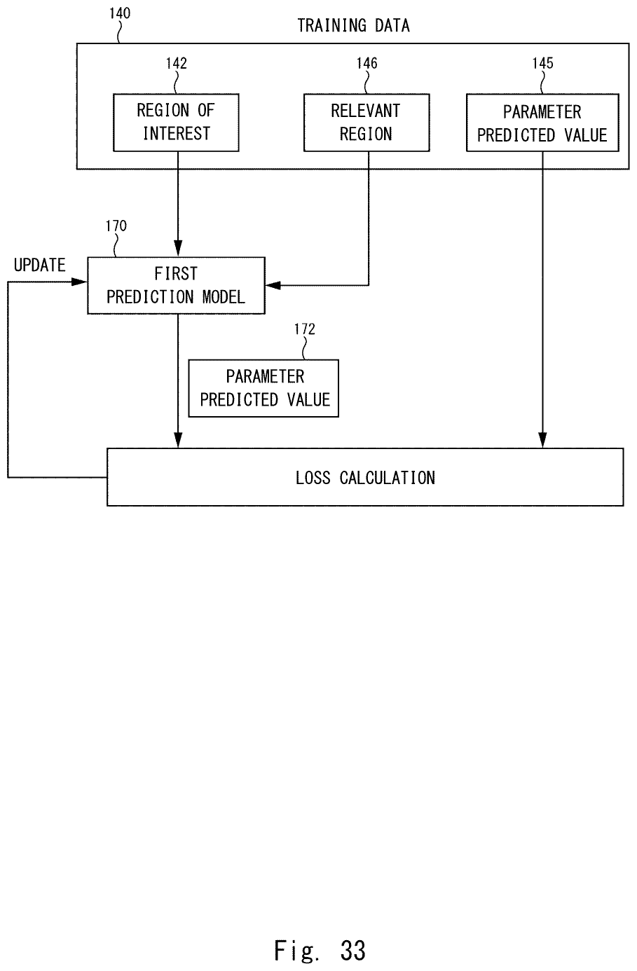

FIG. 33 is a diagram illustrating the training of the first prediction model 170 using a relevant region. The training data 140 includes the ROI 142, the relevant region 146, and the parameter predicted value 145. The training apparatus inputs the ROI 142 and the relevant region 146 into the first prediction model 170 to obtain the parameter predicted value 172. The training apparatus calculates a loss based on the parameter predicted values 172 and 145, and updates each parameter of the first prediction model 170 based on the calculated loss.

A second feature value calculated from the relevant region 14 may be input into the gingival state index value calculation model 50 or the first prediction model 170. In the case where the second feature value is calculated using the second feature value calculation model, the training apparatus further trains the second feature value calculation model.

The second feature value calculation model can be trained in the same manner as in the training of the first feature value calculation model 40. For example, in the training of the gingival state index value calculation model 50, the training apparatus inputs the second feature value calculated from the relevant region 146 by the second feature value calculation model into the gingival state index value calculation model 50, instead of inputting the relevant region 146 into the gingival state index value calculation model 50. The training apparatus calculates a loss based on the gingival state index value 20 output from the gingival state index value calculation model 50 and the gingival state index value 144, and updates each parameter of the gingival state index value calculation model 50 and second feature value calculation model based on the calculated loss.

Similarly, for example, in the training of the first prediction model 170, the training apparatus inputs the second feature value calculated from the relevant region 146 by the second feature value calculation model into the first prediction model 170, instead of inputting the relevant region 146 into the first prediction model 170. The training apparatus calculates a loss based on the parameter predicted values 172 output from the first prediction model 170 and the parameter predicted value 145, and updates each parameter of the first prediction model 170 and second feature value calculation model based on the calculated loss.

In the case where the second feature value calculation model is trained independently, the second feature value calculation model is trained using training data including the relevant region and a ground truth second feature value. The training apparatus calculates a loss based on the second feature value obtained by inputting the relevant region indicated in the training data into the second feature value calculation model and the ground truth second feature value indicated in the training data. The training apparatus then updates each parameter of the second feature value calculation model based on the calculated loss.

The second feature value calculation model may not be used in calculating the second feature value. In this case, the gingival state index value calculation model 50 and the first prediction model 170 are trained using the second feature value calculated from the relevant region 146 with a predetermined algorithm.

In calculating the gingival state index value 20, the attribute information of the subject person may be used. In the case where the attribute information is used to calculate the gingival state index value 20, the training apparatus trains each model with the training data 140 including the attribute information.

### <<Training of Model for Use in Calculation of Alveolar Bone Resorption Index Value 30>>

In calculating the alveolar bone resorption index value 30, the alveolar bone resorption index value calculation model 70 and the second prediction model 180 are used. In the case where the alveolar bone resorption index value calculation model 70 is used, the training apparatus trains the alveolar bone resorption index value calculation model 70.

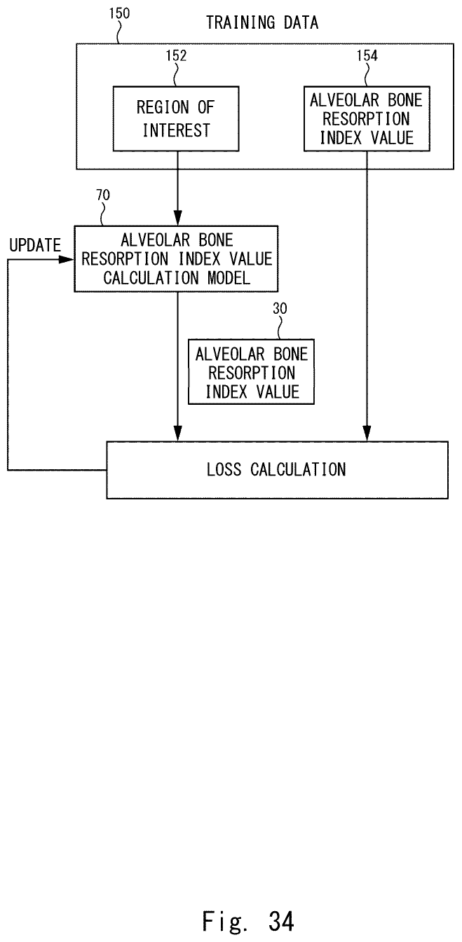

FIG. 34 is a diagram illustrating the training of the alveolar bone resorption index value calculation model 70. Training data 150 includes an ROI 152 and an alveolar bone resorption index value 154. The ROI 152 is a 3D region including a tooth of interest and periodontium of the tooth of interest in the same manner as in the ROI 12. The alveolar bone resorption index value 154 is a ground truth alveolar bone resorption index value to be calculated from the ROI 152 by the alveolar bone resorption index value calculation model 70.

The alveolar bone resorption index value 154 is calculated, for example, using an X-ray image of the tooth of interest and periodontium of the tooth included in the ROI 152. The alveolar bone resorption index value 154 may be a one-dimensional index obtained from one-dimensional data such as a distance, a two-dimensional index obtained from two-dimensional data such as a resorbed alveolar bone area, or a 3D index obtained from 3D data such as a resorbed alveolar bone volume.

The training apparatus inputs the ROI 152 into the alveolar bone resorption index value calculation model 70 to obtain the alveolar bone resorption index value 30. The training apparatus calculates a loss based on the alveolar bone resorption index values 30 and 154, and updates each parameter of the alveolar bone resorption index value calculation model 70 based on the calculated loss.

The training apparatus repeatedly updates the alveolar bone resorption index value calculation model 70 using a plurality of training data 150. Thus, the training apparatus obtains the alveolar bone resorption index value calculation model 70 that has been trained. The trained alveolar bone resorption index value calculation model 70 is then used by the index value calculation apparatus 2000.

As described above, the index value calculation apparatus 2000 may include the alveolar bone resorption index value calculation model 70 for each tooth or tooth part. In this case, the training apparatus trains each alveolar bone resorption index value calculation model 70 prepared for each tooth or tooth part.

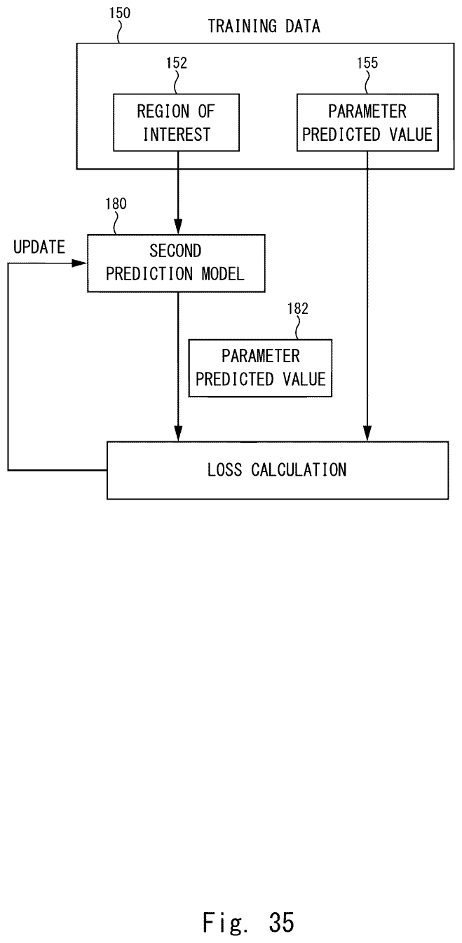

In the case where the second prediction model 180 is used to calculate the alveolar bone resorption index value 30, the training apparatus trains the second prediction model 180. FIG. 35 is a diagram illustrating the training of the second prediction model 180. The training data 150 includes the ROI 152 and a parameter predicted value 155. The parameter predicted value 155 indicates a ground truth predicted value to be output by the second prediction model 180. Suppose that the second prediction model 180 is a model that predicts the position of the root apex. In this case, the parameter predicted value 155 indicates an actual measured value of the position of the root apex in the ROI 152.

The training apparatus inputs the ROI 152 into the second prediction model 180 to obtain a parameter predicted value 182. The training apparatus calculates a loss based on the parameter predicted values 182 and 155, and updates each parameter of the second prediction model 180 based on the calculated loss.

The training apparatus repeatedly updates the second prediction model 180 using a plurality of training data 150. Thus, the training apparatus obtains the second prediction model 180 that has been trained. The trained second prediction model 180 is then used by the index value calculation apparatus 2000.

As described above, a plurality of parameters such as the position of the CEJ, the position of the root apex, and the position of the alveolar crest may be used to calculate the alveolar bone resorption index value 30. Therefore, for each parameter, the training apparatus trains the second prediction model 180 for use in predicting a value of the parameter. For example, the second prediction model 180 that predicts the position of the CEJ is trained using the training data 150 indicating an actual measured value of the position of the CEJ as the parameter predicted value 155. Furthermore, the second prediction model 180 that predicts the position of the root apex is trained using the training data 150 indicating the actual measured value of the position of the root apex as the parameter predicted value 155.

Furthermore, the index value calculation apparatus 2000 may include the second prediction model 180 for the same parameter, for each tooth or tooth part. In this case, the training apparatus trains each second prediction model 180 prepared for each tooth or tooth part.

In calculating the alveolar bone resorption index value 30, the third feature value calculation model 60 may be used. In this case, the training apparatus also trains the third feature value calculation model 60. The training of the third feature value calculation model 60 may be performed together with the training of the alveolar bone resorption index value calculation model 70 and the second prediction model 180, or may be performed independently of the training of the alveolar bone resorption index value calculation model 70 and the second prediction model 180.

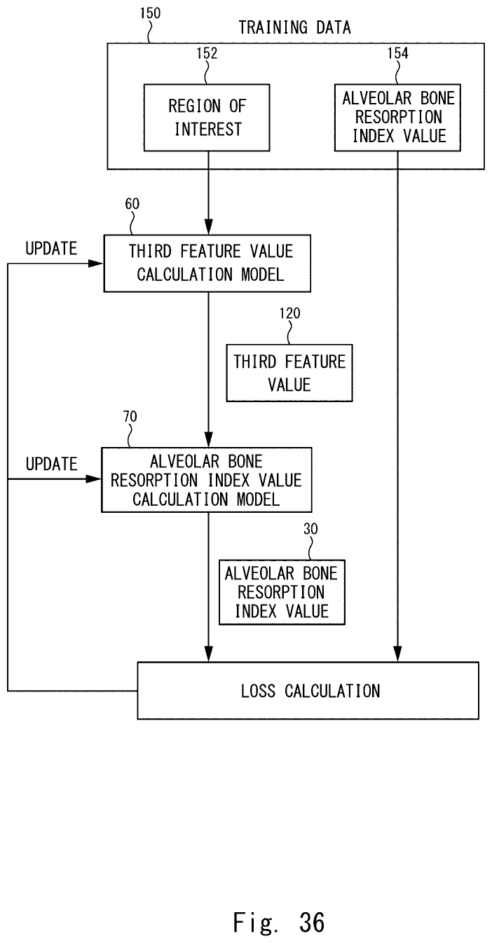

FIG. 36 is a diagram illustrating training of the third feature value calculation model 60 and the alveolar bone resorption index value calculation model 70. The training apparatus inputs the ROI 152 into the third feature value calculation model 60 to obtain the third feature value 120. Furthermore, the training apparatus inputs the third feature value 120 output from the third feature value calculation model 60 into the alveolar bone resorption index value calculation model 70 to obtain the alveolar bone resorption index value 30. The training apparatus calculates a loss based on the alveolar bone resorption index values 30 and 154, and updates each parameter of the third feature value calculation model 60 and alveolar bone resorption index value calculation model 70 based on the calculated loss.

The training apparatus repeatedly updates the third feature value calculation model 60 and the alveolar bone resorption index value calculation model 70 using a plurality of training data 150. Thus, the training apparatus obtains the third feature value calculation model 60 and alveolar bone resorption index value calculation model 70 that have been trained. The trained third feature value calculation model 60 and the trained alveolar bone resorption index value calculation model 70 are then used by the index value calculation apparatus 2000.

A method of training the third feature value calculation model 60 and the second prediction model 180 together is the same as the method of training the third feature value calculation model 60 and the alveolar bone resorption index value calculation model 70 together. That is, the training apparatus inputs the ROI 152 into the third feature value calculation model 60 to obtain the third feature value 120. Furthermore, the training apparatus inputs the third feature value 120 output from the third feature value calculation model 60 into the second prediction model 180 to obtain the parameter predicted value 182. The training apparatus calculates a loss based on the parameter predicted values 182 and 155, and updates each parameter of the third feature value calculation model 60 and second prediction model 180 based on the calculated loss.

In the case where the third feature value calculation model 60 is trained independently, training data including the ROI, and the ground truth third feature value is used. The training apparatus calculates a loss based on the third feature value 120 obtained by inputting the ROI 12 indicated in the training data into the third feature value calculation model 60 and the ground truth third feature value indicated in the training data. Then, the training apparatus updates each parameter of the third feature value calculation model 60 based on the calculated loss.

As described above, the third feature value calculation model 60 may not be used in calculating the third feature value 120. In this case, the third feature value 120 calculated from the ROI 152 with a predetermined algorithm is used to train the alveolar bone resorption index value calculation model 70 and the second prediction model 180.

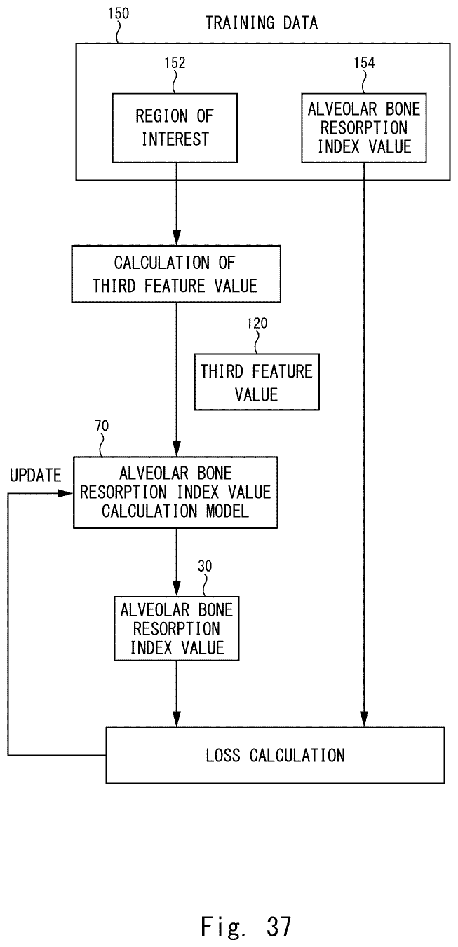

FIG. 37 is a diagram illustrating the training of the alveolar bone resorption index value calculation model 70 into which the third feature value 120 calculated with a predetermined algorithm is input. The training apparatus analyzes the ROI 152 with the predetermined algorithm to calculate the third feature value 120. The training apparatus inputs the calculated third feature value 120 into the alveolar bone resorption index value calculation model 70 to obtain the alveolar bone resorption index value 30. The training apparatus calculates a loss based on the alveolar bone resorption index values 30 and 154, and updates each parameter of the alveolar bone resorption index value calculation model 70 based on the calculated loss.

The second prediction model 180 can be trained in the same manner. Specifically, the training apparatus analyzes the ROI 152 with the predetermined algorithm to calculate the third feature value 120, and inputs the third feature value 120 into the second prediction model 180. The training apparatus calculates a loss based on the parameter predicted value 182 output from the second prediction model 180 and the parameter predicted value 155, and updates the parameter of the second prediction model 180 based on the calculated loss.

In the case where the relevant region 14 is used to calculate the alveolar bone resorption index value 30, the training data 150 including the relevant region is used to train the alveolar bone resorption index value calculation model 70 and the second prediction model 180.

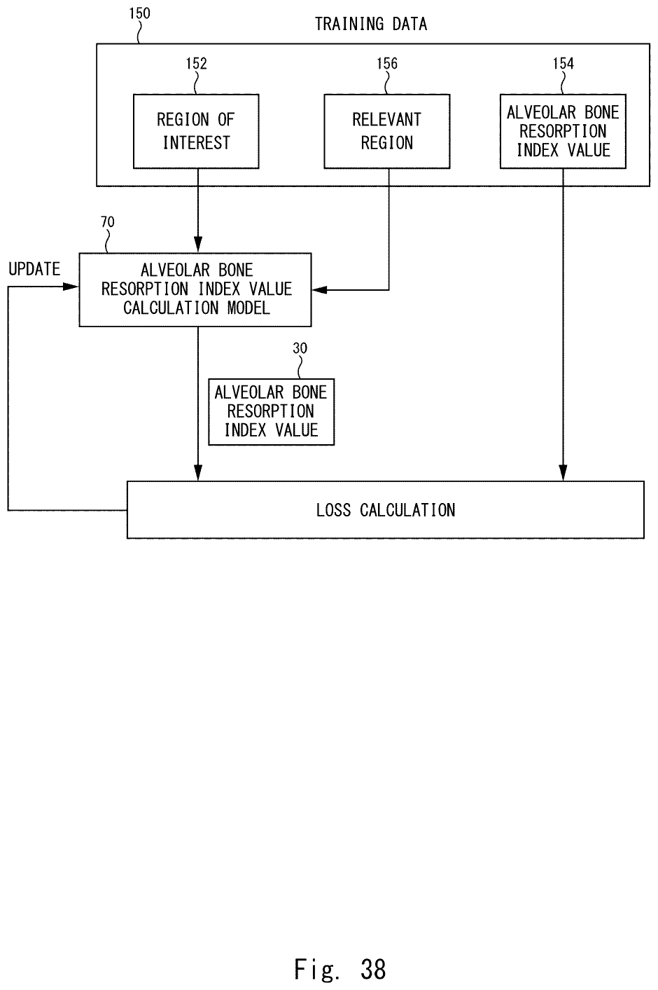

FIG. 38 is a diagram illustrating the training of the alveolar bone resorption index value calculation model 70 using a relevant region. The training data 150 includes the ROI 152, a relevant region 156, and the alveolar bone resorption index value 154. The relevant region 156 is a relevant region corresponding to the ROI 152.

The training apparatus inputs the ROI 152 and the relevant region 156 into the alveolar bone resorption index value calculation model 70 to obtain the alveolar bone resorption index value 30. The training apparatus calculates a loss based on the alveolar bone resorption index values 30 and 154, and updates each parameter of the alveolar bone resorption index value calculation model 70 based on the calculated loss.

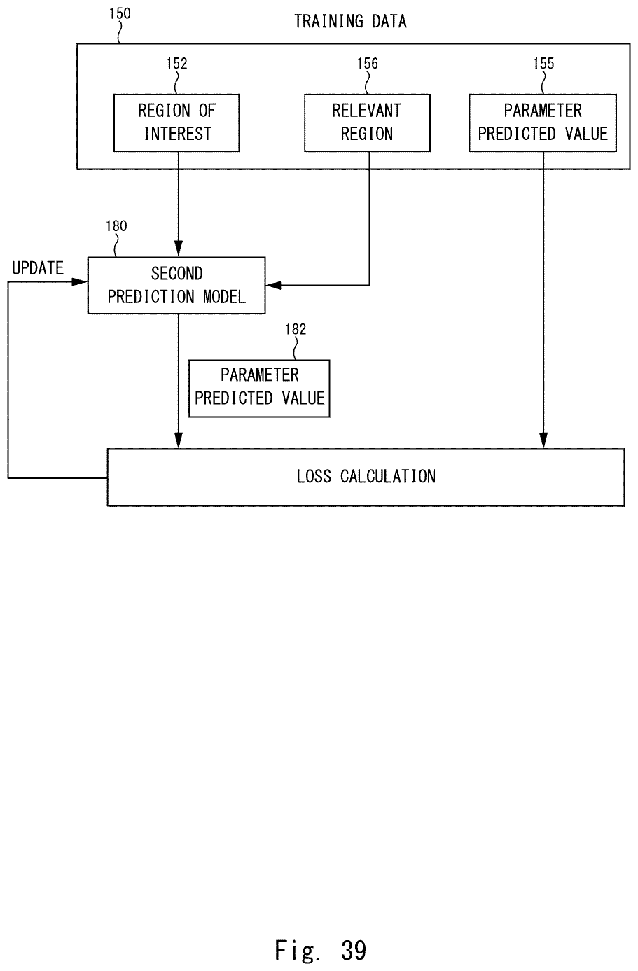

FIG. 39 is a diagram illustrating training of the second prediction model 180 using a relevant region. The training data 150 includes the ROI 152, the relevant region 156, and the parameter predicted value 155. The training apparatus inputs the ROI 152 and the relevant region 156 into the second prediction model 180 to obtain the parameter predicted value 182. The training apparatus calculates a loss based on the parameter predicted values 182 and 155, and updates each parameter of the second prediction model 180 based on the calculated loss.

A fourth feature value calculated from the relevant region 14 may be input into the alveolar bone resorption index value calculation model 70 and the second prediction model 180. When the fourth feature value is calculated using a fourth feature value calculation model, the training apparatus further trains the fourth feature value calculation model.

The fourth feature value calculation model can be trained in the same manner as in the training of the third feature value calculation model 60. For example, in the training of the alveolar bone resorption index value calculation model 70, the training apparatus inputs the fourth feature value calculated from the relevant region 156 by the fourth feature value calculation model into the alveolar bone resorption index value calculation model 70, instead of inputting the relevant region 156 into the alveolar bone resorption index value calculation model 70. The training apparatus calculates a loss based on the alveolar bone resorption index value 30 output from the alveolar bone resorption index value calculation model 70 and the alveolar bone resorption index value 154, and updates each parameter of the alveolar bone resorption index value calculation model 70 and fourth feature value calculation model based on the calculated loss.

Similarly, for example, in the training of the second prediction model 180, the training apparatus inputs the fourth feature value calculated from the relevant region 156 by the fourth feature value calculation model into the second prediction model 180, instead of inputting the relevant region 156 into the second prediction model 180. The training apparatus calculates a loss based on the parameter predicted value 182 output from the second prediction model 180 and the parameter predicted value 155, and updates each parameter of the second prediction model 180 and fourth feature value calculation model based on the calculated loss.

In the case where the fourth feature value calculation model is trained independently, the fourth feature value calculation model is trained using training data including the relevant region and a ground truth fourth feature value. The training apparatus calculates a loss based on the fourth feature value obtained by inputting the relevant region indicated in the training data into the fourth feature value calculation model and the ground truth fourth feature value indicated in the training data. The training apparatus then updates each parameter of the fourth feature value calculation model based on the calculated loss.

The fourth feature value calculation model may not be used in calculating the fourth feature value. In this case, the alveolar bone resorption index value calculation model 70 and the second prediction model 180 are trained using the fourth feature value calculated from the relevant region 156 with a predetermined algorithm.

In calculating the alveolar bone resorption index value 30, attribute information of the subject person may be used. When the attribute information is used to calculate the alveolar bone resorption index value 30, the training apparatus trains each model using the training data 150 including the attribute information.

### <<Verification of Accuracy of Model>>

The training apparatus may use verification data that has the same structure as the training data, to verify the accuracy of the model. For example, for verification of a model for use in calculating the gingival state index value 20, verification data that has the same structure as in the training data 140 is used.

The training apparatus calculates a difference between the gingival state index value 20 calculated using the verification data and the ground truth gingival state index value indicated in the verification data, and determines whether the accuracy of the model is sufficiently high based on the difference. For example, the training apparatus determines that prediction using the model is correct if the ground truth gingival state index value falls within a predetermined numerical range whose center value is the gingival state index value 20 calculated using the model (e.g., ±10% range of gingival state index value 20). On the other hand, it is determined that the prediction using the model is incorrect if the ground truth gingival state index value does not fall within the predetermined numerical range.

For example, the training apparatus makes the determination on each of a plurality of verification data, and determines that the accuracy of the model is sufficiently high if a percentage of the determination that the prediction using the model is correct is equal to or more than a threshold value. On the other hand, it is determined that the accuracy of the model is not sufficiently high if the percentage of the determination that the prediction is correct is less than the threshold value. If it is determined that the accuracy of the model is not sufficiently high, the training apparatus further trains the model for use in calculating the gingival state index value 20, for example, using additional training data 140. In another example, the training apparatus may increase or decrease types of feature values calculated from the ROI or the relevant region. In another example, the training apparatus may change the type of model.

Although the verification of the accuracy of the model that calculates the gingival state index value 20 has been described here, accuracy of the model that calculates the alveolar bone resorption index value 30 can be verified in the same manner.

While the present disclosure has been particularly shown and described with reference to example embodiments thereof, the present disclosure is not limited to these example embodiments. It will be understood by those of ordinary skill in the art that various changes in form and details may be made therein without departing from the spirit and scope of the present disclosure as defined by the claims. And each embodiment can be appropriately combined with at least one of embodiments.

The program includes instructions (or software codes) that, when loaded into a computer, cause the computer to perform one or more of the functions described in the embodiments. The program may be stored in a non-transitory computer readable medium or a tangible storage medium. By way of example, and not a limitation, non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray disc or other types of optical disc storage, and magnetic cassettes, magnetic tape, magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

Each of the drawings or figures is merely an example to illustrate one or more example embodiments. Each figure may not be associated with only one particular example embodiment, but may be associated with one or more other example embodiments. As those of ordinary skill in the art will understand, various features or steps described with reference to any one of the figures can be combined with features or steps illustrated in one or more other figures, for example, to produce example embodiments that are not explicitly illustrated or described. Not all of the features or steps illustrated in any one of the figures to describe an example embodiment are necessarily essential, and some features or steps may be omitted. The order of the steps described in any of the figures may be changed as appropriate.

What is claimed is:

1. A non-transitory computer-readable medium storing a program causing a computer to execute:
acquiring intraoral three-dimensional data that represents a tooth of a subject person and periodontium of the tooth; and
calculating a gingival state index value of the subject person, an alveolar bone resorption index value of the subject person, or both thereof as a state index value of the subject person by using the intraoral three-dimensional data,
wherein the gingival state index value of the subject person is an index value related to a state of a gingival of the subject person,
wherein the alveolar bone resorption index value of the subject person is an index value related to resorption of an alveolar bone of the subject person, and
wherein the state index value is an index value related to a state of teeth of the subject person.

2. The medium according to claim 1,
wherein the calculation of the state index value includes:
calculating a predicted value of each of one or more parameters required for the calculation of the state index value by using a region of interest, the region of interest being a three-dimensional region that is included in the intraoral three-dimensional data and that includes all or a part of a tooth of interest and periodontium of the tooth of interest, the tooth of interest being a tooth regarding which the state index value is to be calculated; and
calculating the state index value based on the calculated predicted value.

3. The medium according to claim 2, comprising:
a prediction model that is trained to output the predicted value of the parameter in response to an input of the region of interest,
wherein the calculation of the state index value includes:
inputting the region of interest into the prediction model to calculate the predicted value of the parameter; and
calculating the state index value using the calculated predicted value of the parameter.

4. The medium according to claim 2, comprising:
a prediction model that is trained to output the predicted value of the parameter in response to an input of the region of interest and a relevant region, the relevant region being a three-dimensional region that is included in the intraoral three-dimensional data and that is relevant to the region of interest,
wherein the calculation of the state index value includes:
extracting the relevant region corresponding to the region of interest from the intraoral three-dimensional data;
inputting the region of interest and the extracted relevant region into the prediction model to calculate the predicted value of the parameter; and
calculating the state index value using the calculated predicted value of the parameter.

5. The medium according to claim 2, comprising:
a prediction model that is trained to output the predicted value of the parameter in response to an input of a feature value related to the tooth of interest,
wherein the calculation of the state index value includes:
calculating the feature value from the region of interest;
inputting the calculated feature value into the prediction model to calculate the predicted value of the parameter; and
calculating the state index value using the calculated predicted value of the parameter.

6. The medium according to claim 1, comprising:
an index value calculation model that is trained to output the state index value in response to an input of a region of interest,
wherein the region of interest is a three-dimensional region that is included in the intraoral three-dimensional data and that includes all or a part of a tooth of interest and periodontium of the tooth of interest, the tooth of interest being a tooth regarding which the state index value is to be calculated, and
wherein the calculation of the state index value includes inputting the region of interest into the index value calculation model to calculate the state index value.

7. The medium according to claim 1, comprising:
an index value calculation model that is trained to output the state index value in response to an input of a region of interest and a relevant region,
wherein the region of interest is a three-dimensional region that is included in the intraoral three-dimensional data and that includes all or a part of a tooth of interest and periodontium of the tooth of interest, the tooth of interest being a tooth regarding which the state index value is to be calculated,
wherein the relevant region is a three-dimensional region that is included in the intraoral three-dimensional data and that is relevant to the region of interest, and
wherein the calculation of the state index value includes:
extracting the relevant region corresponding to the region of interest from the intraoral three-dimensional data; and
inputting the region of interest and the extracted relevant region into the index value calculation model to calculate the state index value.

8. The medium according to claim 1, comprising:
an index value calculation model that is trained to output the state index value in response to an input of a feature value related to a tooth of interest regarding which the state index value is to be calculated,
wherein the calculation of the state index value includes:
calculating the feature value from a region of interest, the region of interest being a three-dimensional region that is included in the intraoral three-dimensional and that includes all or a part of the tooth of interest and periodontium of the tooth of interest; and
inputting the calculated feature value into the index value calculation model to calculate the state index value.

9. The medium according to claim 4,
wherein the relevant region is a three-dimensional region proximal to the region of interest, a three-dimensional region located at a position symmetrical to the region of interest via a tooth centerline of the tooth of interest as a reference, or a three-dimensional region located at a position symmetrical to the region of interest via an oral midline as a reference.

10. The medium according to claim 5,
wherein the feature value related to the tooth of interest represents a shape of the tooth of interest, a size of the tooth of interest, a tone of the tooth of interest, smoothness of the tooth of interest, a distance between the tooth of interest and a tooth adjacent to the tooth of interest, presence or absence of exposure of a cemento-enamel junction in the tooth of interest, a color of gingiva around the tooth of interest, a change in color density of the gingiva depending on a position, a shape of the gingiva, a surface smoothness of the gingiva, a distance between the surface of the gingiva and the surface of the tooth of interest, a surface area of the gingiva, a volume of the gingiva, a distance between gingival alveolar mucosal border of the gingiva and gingival margin, a shape of gingival papilla of the gingiva, a surface area of the gingival papilla of the gingiva, a volume of the gingival papilla of the gingiva, or a gingival papilla height of the gingiva.

11. The medium according to claim 1, comprising using the gingival state index value, the alveolar bone resorption index value, or both thereof for determining presence or absence of a periodontal-related disease, for determining a state of the periodontal-related disease, or determining whether to recommend a visit to dentist

12. An index value calculation apparatus comprising:
at least one memory that is configured to store instructions; and
at least one processor that is configured to execute the instructions to:
acquire intraoral three-dimensional data that represents a tooth of a subject person and periodontium of the tooth; and
calculate a gingival state index value of the subject person, an alveolar bone resorption index value of the subject person, or both thereof as a state index value of the subject person by using the intraoral three-dimensional data,
wherein the gingival state index value of the subject person is an index value related to a state of a gingival of the subject person,
wherein the alveolar bone resorption index value of the subject person is an index value related to resorption of an alveolar bone of the subject person, and
wherein the state index value is an index value related to a state of teeth of the subject person.
